(19) United States
(12) Patent Application Publication
(10) Pub. No.: US 2021/0301304 A1
(43) Pub. Date: Sep. 30, 2021

(54) GLUCOCEREBROSIDASE GENE THERAPY

(71) Applicant: UCL BUSINESS LTD, London (GB)

(72) Inventors: Simon N WADDINGTON, London (GB); Ahad A RAHIM, London (GB); Giulia MASSARO, London (GB)

(21) Appl. No.: 17/250,372

(22) PCT Filed: Jul. 4, 2019
(86) PCT No.: PCT/GB2019/051896

§ 371 Date: Jan. 12, 2021

(30) Foreign Application Priority Data
Jul. 13, 2018 (GB) 1811540.2
Jul. 13, 2018 (GB) 1811541.0
Jan. 25, 2019 (GB) PCT/GB2019/050214

(51) Int. Cl. C12N 15/86; C12N 9/24; A61P 3/00; A61K 9/00

(52) U.S. Cl. CPC C12N 15/86; C12N 9/2402; C12Y 302/01045; C12N 2750/14143; A61K 9/0029; A61K 9/0019; A61K 9/0085; A61P 3/00

(57) ABSTRACT

The present invention relates to expression constructs and vectors for the treatment and/or prevention of diseases that are associated with a loss of GBA1 function, such as lysosomal storage disorders including Gaucher disease or Niemann-Pick type C (NPC) disease, and synucleinopathies including Parkinsons disease, dementia with Lewy bodies, multi-system atrophy (MSA) or pure autonomic failure (PAF).

### FIELD OF THE INVENTION

The present invention relates to expression constructs and vectors for the treatment and/or prevention of diseases that are associated with a loss of GBA1 function, such as lysosomal storage disorders including Gaucher disease or Niemann-Pick type C (NPC) disease, and synucleinopathies including Parkinson's disease, dementia with Lewy bodies, multi-system atrophy (MSA) or pure autonomic failure (PAF).

### BACKGROUND OF THE INVENTION

Gaucher disease is a lysosomal storage disorder caused by mutations in the GBA1 gene encoding the enzyme glucocerebrosidase (GCase). Deficiency of GCase causes the accumulation of its substrate glucosylceramide in both visceral organs and the CNS. Enzyme replacement therapy (ERT) is successfully used to ameliorate the visceral pathology, however there is no treatment available for the neurodegeneration. Neuronopathic Gaucher disease (nGD), or Gaucher disease type II, is an inherited acute childhood lethal genetic disease. nGD patients exhibit signs consistent with hindbrain neurodegeneration including neck hyperextension, strabismus and, often, fatal apnea. Neuropathology results in death during early infancy. In adult patients, a milder form of Gaucher disease, Gaucher disease type I, presents with hepatomegaly, splenomegaly and occasional lung and bone disease; this is managed, symptomatically, by ERT. Although ERT has revolutionised the treatment of type I Gaucher disease it has a number of drawbacks. This includes the need for repeated and regular infusions for the duration of the patient's life, which is both difficult and expensive and may fail to treat some tissues, such as the lungs and bones. Skeletal manifestations of Gaucher disease are thought to respond more slowly to ERT than those in the liver and spleen. nGD is untreatable since the enzyme therapy cannot cross the blood brain barrier. Gaucher disease type III is the chronic neuronopathic form of Gaucher disease. Individuals can manifest the first symptoms already in early infancy, however the progression of the disease is slow and lifespan can be prolonged to adulthood. The visceral pathology is extended and present at birth, while the neurological signs can appear before two years of age or manifest later in life. In some cases of chronic neurologic Gaucher disease, dementia and ataxia can be observed in the latest stage of the pathology. The clinical course not always depends on age of onset and rate of progression. Furthermore, a subset of patients can manifest an intermediate phenotype between type II and III, characterised by a late onset and rapid progression of acute neurodegeneration.

Treatments for Gaucher patients can also be used to treat patients with other lysosomal storage disorders. For example, Miglustat is a substrate-reduction therapy (SRT) for Gaucher disease patients, which is also used for Niemann-Pick type C patients for treating progressive neurological complications.

In addition, there is growing literature suggesting that loss of GBA1 function leads to the abnormal accumulation of alpha-synuclein protein in neurons, nerve fibres or glial cells, which in turn leads to synucleinopathies. Examples of synucleinopathies include Parkinson's disease, dementia with Lewy bodies, multi-system atrophy (MSA) and pure autonomic failure (PAF). The mechanistic links between glucocerebrosidase and α-synuclein are unclear, but there appears to be an inverse correlation between the levels of glucocerebrosidase and α-synuclein. Experimental evidence also supports a direct interaction between α-synuclein and glucocerebrosidase.

Recent studies have indicated that there is an increased frequency of mutations in the GBA1 gene among patients with Parkinson's disease. Mutations in the GBA1 gene are some of the most common genetic risk factors for Parkinson's that have been identified. Mutations in the GBA1 gene have also been reported as significant risk factors for Lewy body disorders, such as dementia with Lewy bodies. Studies have also shown that Gaucher-disease-causing GBA variants are significantly associated with MSA cerebellar subtype (MSA-C) patients.

There is thus a need for improved therapies for the treatment of lysosomal storage disorders such as Gaucher or Niemann-Pick type C (NPC) disease. In addition, there is a need for improved therapies for the treatment of synucleinopathies, such as Parkinson's disease, dementia with Lewy bodies, multi-system atrophy (MSA) or pure autonomic failure (PAF).

### SUMMARY OF THE INVENTION

The present invention is based on the creation of an optimised expression construct for expressing the GBA1 gene in cells.

Optimised expression constructs are shown in SEQ ID NOs: 5 to 8; or SEQ ID NOs: 20 to 21. Certain codon optimised GBA sequences encoding GBA1 (SEQ ID NO: 12) are shown in SEQ ID NOs: 13 to 16.

In addition, the invention also relates to optimised gene therapy vectors for expressing the GBA1 gene in cells and plasma. In a preferred embodiment of the invention, the optimised vectors are shown in SEQ ID NOs: 9, 10, 17 and 18.

Constructs of the invention comprise a sequence encoding GBA1 and either a CBA or CAG promoter. Both of these are ubiquitous promoters. Gene therapy with vectors of the invention in GBA1 knockout mouse models of acute neuronopathic Gaucher disease led to increased survival rates with both CBA and CAG, with CAG increasing survival rates to wild-type levels, although the CBA vector-treated animals did not survive to 8 weeks. Vectors containing the neuronal specific synapsin promoter used in previously disclosed Gaucher disease gene therapy treatments performed similarly. Mice surviving to 8 weeks were examined for motor coordination and again CAG and synapsin vectors performed similarly, with vectors being found to normalise motor function, and also exhibited no significant differences in spleen or liver weight compared to wild-type animals. In mice treated with CAG and synapsin vectors, GCase enzyme activity was restored and GCase substrate levels were reduced in brain tissue. Furthermore, mice treated with CAG and synapsin vectors also exhibited reduced astrocyte and lysosome accumulation, together with decreased macrophage activation in brain tissue. In plasma, systemically increased GCase activity was observed with both CAG and CBA vectors but not with the synapsin vector but all three vectors reduced substrate levels. In lung tissue, both CAG and CBA restored GCase activity and reduced substrate levels but synapsin vectors did not. Furthermore, the CAG vector prevented the accumulation of enlarged lysosomes and significantly reduced macrophage activation in the lung. Similar observations were made in liver tissue whereby mice treated with CAG and CBA vectors exhibited increased GCase activity compared to GBA1 knockout mice but synapsin vectors did not. Gene therapy using the CAG and CBA vectors also reduced substrate levels in the liver compared to GBA1 knockout mice. Mice treated with CAG, CBA or synapsin vectors exhibited at least a partial reduction in macrophage activation and decreased the abundance of enlarged lysosomes in the liver. In spleen tissue, both CAG and CBA vectors increased GCase enzyme activity and reduced substrate levels, whereas gene therapy with synapsin vectors had no effect. Gene therapy with the CAG vector also reduced macrophage activation and substrate accumulation in lysosomes of spleen tissue.

Therefore, the vectors of invention are superior to previously used ones in that they are active in a wider range of tissues and hence will be suitable to treat patients suffering from all forms of Gaucher disease, not just those with neuronopathic forms. Even though CAG and CBA are both ubiquitous promoters, CAG however confers increased survival, supramaximal GCase activity in lung tissue whilst also increasing GCase activity in plasma, liver, brain and spleen. Furthermore, CAG gene therapy substantially reduces neuroinflammation in the brain, in addition to reducing macrophage activation and substrate accumulation in lysosomes in the lung, liver, brain and spleen, without compromising neuronal count. Vectors containing CAG are therefore particularly promising in gene therapy approaches to Gaucher disease.

Accordingly, the invention provides:

An expression construct comprising in a 5′ to 3′ direction:

(a) the CBA promoter as shown in SEQ ID NO: 2, or a sequence having at least 90% sequence identity to SEQ ID NO: 2 that retains the ability to express GBA1; and
(b) (i) the GBA1 sequence as shown in SEQ ID NO: 1, or a sequence having at least 70% sequence identity to SEQ ID NO: 1 that retains the functionality of GBA1; or (ii) a GBA1 sequence encoding the polypeptide as shown in SEQ ID NO: 12 or a polypeptide having at least 90% sequence identity to the polypeptide of SEQ ID NO: 12 that retains the functionality of GBA1.

The invention also provides:

An expression construct comprising in a 5′ to 3′ direction:

(a) the CAG promoter as shown in SEQ ID NO: 3, or a sequence having at least 90% sequence identity to SEQ ID NO: 3 that retains the ability to express GBA1; and
(b) (i) the GBA1 sequence as shown in SEQ ID NO: 1, or a sequence having at least 70% sequence identity to SEQ ID NO: 1 that retains the functionality of GBA1; or (ii) a GBA1 sequence encoding the polypeptide as shown in SEQ ID NO: 12 or a polypeptide having at least 90% sequence identity to the polypeptide of SEQ ID NO: 12 that retains the functionality of GBA1.

The invention also provides vectors and viral vectors comprising the expression constructs of the invention. The invention also provides host cells comprising the vectors or viral vectors of the invention. The invention also provides pharmaceutical compositions comprising the vectors of the invention and pharmaceutically acceptable carriers.

The invention also encompasses:

A vector of the invention for use in a method of preventing or treating Gaucher disease; or

a vector of the invention for use in a method of preventing or treating lysosomal storage disorders such as Niemann-Pick disease type C (NPC), or synucleinopathies such as Parkinson's disease, dementia with Lewy bodies, multiple system atrophy and pure autonomic failure (PAF).

The invention also encompasses:

Use of a vector according to the invention in the manufacture of a medicament for the treatment or prevention of Gaucher disease; or

use of a vector according to the invention in the manufacture of a medicament for the treatment or prevention of lysosomal storage disorders such as Niemann-Pick disease type C (NPC), or synucleinopathies such as Parkinson's disease, dementia with Lewy bodies, multiple system atrophy and pure autonomic failure (PAF).

The invention also encompasses:

A method of treating or preventing Gaucher disease in a patient in need thereof, comprising administering a therapeutically effective amount of a vector of the invention to said patient; or

a method of treating or preventing lysosomal storage disorders such as Niemann-Pick disease type C (NPC), or synucleinopathies such as Parkinson's disease, dementia with Lewy bodies, multiple system atrophy and pure autonomic failure (PAF) in a patient in need thereof, comprising administering a therapeutically effective amount of a vector of the invention to said patient.

### BRIEF DESCRIPTION OF THE FIGURES

FIG. 1: Diagram of pAAV.CAG.GBA.WPRE

FIG. 2: Diagram of pAAV.CBA.GBA.WPRE

FIG. 3: Diagram of pAAV.SYN.GBA.WPRE

FIG. 4: Transfection of GBA gene therapy vectors in vitro increases GBA enzymatic activity. Graph showing β-Glucocerebrosidase activity in HEK293T cells and supernatant after transfection with the gene therapy vectors pAAV.CAG.GBA.WPRE, pAAV.CBA.GBA.WPRE, or pAAV.SYN.GBA.WPRE. All transfected cells displayed significantly increased β-Glucocerebrosidase activity compared to the untransduced control.

FIG. 5: Increased survival following GBA1 gene therapy in an acute neuronopathic Gaucher Disease mouse model.

A. Kaplan-Meier survival plots of K14-Inl/Inl KO mice following treatment with 2.4×10¹⁵ vg/kg (n=9) or 3.3×10¹⁴ vg/kg (n=9) AAV9.SYN.GBA1 gene therapy, compared to untreated K14-Inl/Inl KO mice (n=8) and wild-type (WT) animals (n=9). The untreated KO mice die by 2-weeks of age, whereas those animals treated with 2.4×10¹⁵ vg/kg AAV9.SYN.GBA1 display the same survival rate as WT mice. The lower dose group (3.3×10¹⁴ vg/kg AAV9.SYN.GBA1) showed reduced survival with 7 out of the 9 animals surviving until the cull at 8-weeks.

B. Kaplan-Meier survival plot of K14-Inl/Inl KO mice following treatment with 2.4×10¹⁵ vg/kg (n=9) or 3.3×10¹⁴ vg/kg (n=9) AAV9.CAG.GBA1 gene therapy, compared to untreated K14-Inl/Inl KO mice (n=8) and wild-type (WT) animals (n=9). The untreated KO mice die by 2-weeks of age, whereas those animals treated with 2.4×10¹⁵ vg/kg AAV9.CAG.GBA1 display the same survival rate as WT mice. The lower dose group (3.3×10¹⁴ vg/kg AAV9.CAG.GBA1) showed reduced survival with 3 out of the 9 animals surviving until the cull at 8-weeks.

C. Kaplan-Meier survival plot of K14-Inl/Inl KO mice following treatment with 2.4×10¹⁵ vg/kg (n=9) or 3.3×10¹⁴ vg/kg (n=8) AAV9.CBA.GBA1 gene therapy, compared to untreated K14-Inl/Inl KO mice (n=8) and wild-type (WT) animals (n=9). Those animals injected with 3.3×10¹⁴ vg/kg AAV9.CBA.GBA1 died by 2-weeks of age, similarly to the untreated KO mice. Those animals treated with 2.4×10¹⁵ vg/kg AAV9.CBA.GBA1 survived slightly longer, though none of the mice survived until 8-weeks.

FIG. 6: Administration of GBA1 gene therapy normalises motor function and behaviour in animals surviving to 8-weeks. Open field traces representative of animals that survived to 8-weeks. A=WT. B=2.4×10¹⁵ vg/kg AAV2/9.CAG.GBA1. C=2.4×10¹⁵ vg/kg AAV2/9.SYN.GBA1. All traces appear similar, suggesting that animals treated with AAV9.SYN.GBA1 and AAV9.CAG.GBA1 have the same level of motor coordination as the wild-type animals.

FIG. 7: Animals administered GBA1 gene therapy maintain normal spleen weight. Spleen weights adjusted for body weight at 8-weeks from WT; 2.4×10¹⁵ vg/kg SYN.GBA1; and 2.4×10¹⁵ vg/kg CAG.GBA1 groups. The spleen weights of the animals treated with AAV9.SYN.GBA1 and AAV9.CAG.GBA1 were not significantly different from the WT animals. Error bars are mean with SD.

FIG. 8: Animals administered GBA1 gene therapy maintain normal liver weight. Liver weights adjusted for body weight at 8-weeks from WT; 2.4×10¹⁵ vg/kg SYN.GBA1; and 2.4×10¹⁵ vg/kg CAG.GBA1 groups. The liver weights of the animals treated with AAV9.SYN.GBA1 and AAV9.CAG.GBA1. were not significantly different from the WT animals. Error bars are mean with SD.

FIG. 9: Examining GCase enzymatic activity in brain homogenate following GBA1 gene therapy. GCase enzyme activity in brain homogenate for all treatment groups compared to untreated KO mice and WT animals. Error bars are mean with SD. The higher dose groups (2.4×10¹⁵ vg/kg) for SYN.GBA1 and CAG.GBA1 increase GCase activity to levels equivalent or greater than activity observed in the WT animals.

FIG. 10: Examining glucosylceramide substrate levels in brain homogenate following GBA1 gene therapy.

A: GlcCer C16:0 substrate levels in brain homogenate for all treatment groups compared to untreated KO mice and WT animals. Error bars are mean with SD. The higher dose groups (2.4×10¹⁵ vg/kg) for SYN.GBA1 and CAG.GBA1 reduce the GlcCer substrate levels to equivalent as those observed in the WT animals.

B: GlcCer C18:0 substrate levels in brain homogenate for all treatment groups compared to untreated KO mice and WT animals. Error bars are mean with SD. The higher dose groups (2.4×10¹⁵ vg/kg) for SYN.GBA1 and CAG.GBA1 reduce the GlcCer substrate levels to equivalent as those observed in the WT animals.

C: GlcCer C20:0 substrate levels in brain homogenate for all treatment groups compared to untreated KO mice and WT animals. Error bars are mean with SD. The higher dose groups (2.4×10¹⁵ vg/kg) for SYN.GBA1 and CAG.GBA1 reduce the GlcCer substrate levels to equivalent as those observed in the WT animals.

D: GlcCer C22:0 substrate levels in brain homogenate for all treatment groups compared to untreated KO mice and WT animals. Error bars are mean with SD. The higher dose groups (2.4×10¹⁵ vg/kg) for SYN.GBA1 and CAG.GBA1 reduce the GlcCer substrate levels to equivalent as those observed in the WT animals.

E: GlcCer C23:0 substrate levels in brain homogenate for all treatment groups compared to untreated KO mice and WT animals. There is little change in the levels of GlcCer C23:0 between all groups.

F: GlcCer C24:0 substrate levels in brain homogenate for all treatment groups compared to untreated KO mice and WT animals. There is little change in the levels of GlcCer C24:0 between all groups.

FIG. 11: Increased GCase enzymatic activity in plasma following GBA1 gene therapy using CAG and CBA promoters. GCase enzyme activity in plasma for all treatment groups compared to untreated KO mice and WT animals. Error bars are ±SD. The ubiquitous promoters CAG and CBA lead to increased systemic activity of GCase, for both dose groups. The SYN promoter does not increase the systemic activity of GCase above levels observed in the KO animals.

FIG. 12: Examining glucosylceramide substrate levels in the plasma following GBA1 gene therapy. GlcCer 16:0 levels in plasma for all treatment groups compared to untreated KO mice and WT animals. There is accumulation of GlcCer C16:0 levels in the plasma of KO animals and those treated with 3.3×10¹⁴ vg/kg dose of CBA.GBA1. All other groups resemble the wildtype levels.

FIG. 13: GCase enzymatic activity increases in lung homogenate following GBA1 gene therapy using CAG and CBA promoters.

A and B: GCase enzyme activity in lung homogenate for all treatment groups compared to untreated KO mice and WT animals. Error bars are ±SD. The 2.5×10¹⁵ vg/kg dose of CAG.GBA1 led to supramaximal activity of GCase in the lung. The 3.3×10¹⁴ vg/kg dose of CAG.GBA1 and 2.5×10¹⁵ vg/kg dose of CBA.GBA1 both led to increased levels of GCase activity that were similar to the wildtype group (see more clearly in FIG. 9b).

FIG. 14: Examining GCase enzymatic activity in liver homogenate following GBA1 gene therapy. GCase enzyme activity in liver homogenate for all treatment groups compared to untreated KO mice and WT animals. Error bars are ±SD. The 2.5×10¹⁵ vg/kg dose of CAG.GBA1 led to wildtype levels of GCase activity in the liver. The 3.3×10¹⁴ vg/kg dose of CAG.GBA1 and 2.5×10¹⁵ vg/kg dose of CBA.GBA1 also both led to increased levels of GCase activity.

FIG. 15: Examining glucosylceramide substrate levels in the lung following GBA1 gene therapy. Total GlcCer substrate level (total includes C:16, C:18, C:20, C:22, C:23 and C:24 fatty acid compositions) in lung homogenate for all treatment groups compared to untreated KO mice and WT animals. Log 10 scale. Error bars are mean±SD. The 2.5×10¹⁵ vg/kg dose of CAG.GBA1 and CBA.GBA1 led to GlcCer levels similar to wildtype levels. The 3.3×10¹⁴ vg/kg dose of CAG.GBA1 and CBA.GBA1 also led to a reduction in GlcCer substrate levels when compared to the untreated KO group.

FIG. 16: Examining glucosylceramide substrate levels in liver homogenate following GBA1 gene therapy. Total GlcCer substrate level (total includes C:16, C:18, C:20, C:22, C:23 and C:24 fatty acid compositions) in liver homogenate for all treatment groups compared to untreated KO mice and WT animals. Log 10 scale. Error bars are mean±SD. The 2.5×10¹⁵ vg/kg and 3.3×10¹⁴ vg/kg dose of CAG.GBA1 led to GlcCer levels similar to wildtype levels. Both doses of CBA.GBA1 also led to a reduction in GlcCer substrate levels when compared to the untreated KO group.

FIG. 17: Examining GCase enzymatic activity in spleen homogenate following GBA1 gene therapy. GCase enzyme activity in spleen homogenate for all treatment groups compared to untreated KO mice and WT animals. Error bars are ±SD. The 2.5×10¹⁵ vg/kg dose of CAG.GBA1 led to levels of GCase activity in the spleen that were similar to WT animals. The 3.3×10¹⁴ vg/kg dose of CAG.GBA1 and 2.5×10¹⁵ vg/kg dose of CBA.GBA1 also both led to increased levels of GCase activity in the spleen.

FIG. 18: Examining glucosylceramide substrate levels in the spleen following GBA1 gene therapy. Total GlcCer substrate level (total includes C:16, C:18, C:20, C:22, C:23 and C:24 fatty acid compositions) in spleen homogenate for all treatment groups compared to untreated KO mice and WT animals. Log 10 scale. Error bars are mean±SD. The 2.5×10¹⁵ vg/kg dose of CAG.GBA1 and CBA.GBA1 led to GlcCer levels similar to wildtype levels. The 3.3×10¹⁴ vg/kg dose of CAG.GBA1 and CBA.GBA1 also led to a reduction in GlcCer substrate levels when compared to the untreated KO group.

FIG. 19: Examining astrocyte marker, glial fibrillary acidic protein (GFAP), in brain tissue following treatment with GBA1 gene therapy. GFAP expression in brain tissue for all treatment groups compared to untreated knockout (KO) mice and wildtype (WT) animals. The higher (2.5×10¹⁵ vg/kg) dose of SYN.GBA1 and CAG.GBA1 reduced the expression of GFAP to similar levels observed in the wildtype group in the Ventral post medial/ventral post lateral thalamic nuclei (VPM/VPL) region (A); Gigantocellular nuclei (Gi) region (B); Somato-barrel field 1 (S1BF) region (C). The lower dose group (3.3×10¹⁴ vg/kg) of SYN.GBA1 and CAG.GBA1, as well as both dose groups for CBA.GBA1, had less effect on the expression of GFAP when compared to tissue from the untreated KO animals.

FIG. 20: Examining macrophage marker CD68 in brain tissue following treatment with GBA1 gene therapy. CD68 expression in brain tissue for all treatment groups was compared to untreated KO mice and WT animals. The higher (2.5×10¹⁵ vg/kg) dose of SYN.GBA1 and CAG.GBA1 reduce the expression of CD68 to similar levels observed in the WT group in S1BF region (A); Gi region (B); VPM/VPL region (C). The lower dose group (3.3×10¹⁴ vg/kg) of SYN.GBA1 and CAG.GBA1, as well as both dose groups for CBA.GBA1, had less effect on the expression of GFAP when compared to the untreated KO tissue.

FIG. 21: Examining macrophage marker CD68 expression in liver tissue following treatment with GBA1 gene therapy. Liver sections were stained for the macrophage marker CD68 to detect the presence of activated macrophages. The 2.5×10¹⁵ vg/kg dose of SYN.GBA1 and CAG.GBA1 led to wildtype-like appearance of CD68 expression in the liver. The lower doses of SYN.GBA1 and CAG.GBA1, and both dose groups for CBA.GBA1, partially reduce CD68 expression when compared to the untreated KO group.

FIG. 22: Examining macrophage marker CD68 in lung tissue following treatment with GBA1 gene therapy. Lung sections were stained for the macrophage marker CD68 to detect the presence of activated macrophages. All treatment groups had an effect on the expression of CD68 in the lung. Treatment with both 2.5×10¹⁵ vg/kg and 3.3×10¹⁴ vg/kg CAG.GBA1 had the greatest effect, significantly reducing CD68 expression; CD68 positive cells were smaller and with morphology comparable to wild-type.

FIG. 23: Examining macrophage marker CD68 in spleen tissue following treatment with GBA1 gene therapy. Spleen sections were stained for the macrophage marker CD68 to detect the presence of activated macrophages. Macrophages are diffuse in physiological condition in wild-type spleen tissue, particularly in the red pulp regions (Red Pulp Macrophages). Therefore, scattered CD68-positive stained was observed through-out the sections. Untreated KO mice developed abnormal accumulation of macrophages within the white pulp, where several enlarged CD68-positive cells were identified. Administration of 2.5×10¹⁵ vg/kg CAG.GBA1 did not lead to formation of enlarged macrophages in the white pulp. Several enlarged CD68-positive cells were identified in the white pulp tissue in tissue from animals treated with SYN.GBA1 and CBA.GBA1 at both doses.

FIG. 24: Examining lysosomal marker Lamp1 in brain tissue VPM following treatment with GBA1 gene therapy. The lysosome-associated membrane protein 1 (LAMP1) marker was used to assess enlarged lysosomes resulting from pathological accumulation of GlcCer substrate in the brain. All the regions of untreated KO brains were affected by intense lysosome accumulation, presenting intense LAMP1 staining. IV administration of 2.5×10¹⁵ vg/kg SYN.GBA1 and 2.5×10¹⁵ vg/kg CAG.GBA1 resulted in reduction of cellular lysosomal content comparable to wild-type levels in all analysed brain regions; S1BF region (A); Gi region (B); VPM/VPL region (C).

FIG. 25: Examining lysosomal marker Lamp1 in liver tissue following treatment with GBA1 gene therapy. Lamp1 staining was used to assess the presence of enlarged lysosomes within liver tissue. Liver sections from untreated KO mice presented numerous swollen and dark-stained organelle, while the wild-type tissue showed small punctated staining, index of resting regular lysosomes. IV administration of both doses of CAG.GBA1 prevented accumulation of enlarged lysosome and LAMP1 staining was comparable to wild-type tissue. All other treatment groups demonstrated partial reduction of Lamp1.

FIG. 26: Examining lysosomal marker Lamp1 in lung tissue following treatment with GBA1 gene therapy. The presence of enlarged lysosomes was assessed by Lamp1 staining. Untreated KO mice developed engorged lysosomes, represented in the images as dark large stained dots. The normal WT tissue was characterized by smaller round-shaped stained resident organelles. IV administration of 2.5×10¹⁵ vg/kg CAG.GBA1 prevented accumulation of enlarged lysosomes and lung tissue was comparable to the WT, with small punctate staining. In the lung sections from mice administered with 3.3×10¹⁴ vg/kg CAG.GBA1 only a few mildly enlarged lysosomes were present.

FIG. 27: Examining lysosomal marker Lamp1 in spleen tissue following treatment with GBA1 gene therapy. Spleen tissue from untreated KO mice was severely disrupted, characterized by numerous enlarged lysosomes particularly within the white pulp regions. Wild-type tissue was homogenously stained, and physiological levels of LAMP1 staining were detected. Lysosomes were depicted as small round-shaped puncta. Administration of both doses of CAG.GBA1 led to reduced number and size of LAMP1-positive organelles compared to KO tissue. All other treatment groups had a partial effect on Lamp1 expression in spleen tissue.

FIG. 28: Examining the cortical thickness of K14 In/Inl Gba1 KO animals treated with GBA1 gene therapy. The thickness of the somato-barrel field region 1 (SiBF) cortical region was measured with Stereo Investigator software on Nissl stained sections. Treatment of KO animals with SYN.GBA1 and CAG.GBA1 gene therapy maintained the cortical region to a similar thickness as observed for WT animals. Error bars±SD.

FIG. 29: Examining the neuronal count of the S1BF cortical region of K14 Inl/Inl Gba1 KO animals treated with GBA1 gene therapy. Neuronal counts were calculated using stereology. The number of neurons in the SiBF cortical region demonstrated that brains from KO mice treated with SYN.GBA1 and CAG.GBA1 were not subject to neuronal loss (data presented as average t SD).

FIG. 30: Examining the vector copy number in brain tissue from K14-Inl/Inl Gba1 KO animals treated with GBA1 gene therapy. The higher dose groups (2.5×10¹⁵ vg/kg) of SYN.GBA1, CAG.GBA1 and CBA.GBA1 all demonstrated a higher vector copy number than the lower dose (3.3×10¹⁴ vg·kg) treatment groups in brain tissue, as measured by qPCR. Error bars are average t SD.

FIG. 31: Examining the vector copy number in liver tissue from K14-Inl/Inl Gba1 KO animals treated with GBA1 gene therapy. The higher dose groups (2.5×10¹⁵ vg/kg) of SYN.GBA1, CAG.GBA1 and CBA.GBA1 all demonstrated a higher vector copy number than the lower dose (3.3×10¹⁴ vg·kg) treatment groups in liver tissue, as measured by qPCR. Error bars are average t SD.

FIG. 32: Examining the vector copy number in lung tissue from K14-Inl/Inl Gba1 KO animals treated with GBA1 gene therapy. The higher dose groups (2.5×10¹⁵ vg/kg) of SYN.GBA1, CAG.GBA1 and CBA.GBA1 all demonstrated a higher vector copy number than the lower dose (3.3×10¹⁴ vg·kg) treatment groups in lung tissue, as measured by qPCR.

Error bars are average t SD.

FIG. 33: Examining the vector copy number in spleen tissue from K14-Inl/Inl Gba1 KO animals treated with GBA1 gene therapy. The higher dose groups (2.5×10¹⁵ vg/kg) of CAG.GBA1 and CBA.GBA1 demonstrated a higher vector copy number than the higher dose group for SYN.GBA1. Error bars are average t SD.

FIG. 34 Examining the vector copy number in heart tissue from K14-Inl/Inl Gba1 KO animals treated with GBA1 gene therapy. The higher dose groups (2.5×10¹⁵ vg/kg) of SYN.GBA1, CAG.GBA1 and CBA.GBA1 all demonstrated a higher vector copy number than the lower dose (3.3×10¹⁴ vg·kg) treatment groups in heart tissue, as measured by qPCR. Error bars are average t SD.

### BRIEF DESCRIPTION OF THE SEQUENCES

SEQ ID NO: 1—human GBA1 coding sequence (NCBI gene ID 2629)

SEQ ID NO: 2—the CBA promoter sequence

SEQ ID NO: 3—the CAG promoter sequence

SEQ ID NO: 4—the WPRE sequence

SEQ ID NO: 5—the CBA promoter sequence operably linked to the GBA1 coding sequence

SEQ ID NO: 6—CAG promoter sequence operably linked to the GBA1 coding sequence

SEQ ID NO: 7—the CBA promoter sequence operably linked to the GBA1 coding sequence and the WPRE sequence

SEQ ID NO: 8—CAG promoter sequence operably linked to the GBA1 coding sequence and the WPRE sequence

SEQ ID NO: 9—pAAV.CBA.hGBA1.WPRE.hGHpA vector

SEQ ID NO: 10—pAAV.CAG.hGBA1.WPRE.hGHpA vector

SEQ ID NO: 11—pAAV.hSYN.hGBA1.WPRE.hGHpA vector

SEQ ID NO: 12—the GBA1 polypeptide sequence

SEQ ID NO: 13—a codon optimised GBA1 sequence

SEQ ID NO: 14—a codon optimised GBA1 sequence

SEQ ID NO: 15—a codon optimised GBA1 sequence

SEQ ID NO: 16—a codon optimised GBA1 sequence

SEQ ID NO: 17—pAAV.CBA.hGBA1.WPRE.hGHpA vector

SEQ ID NO: 18—pAAV.CAG.hGBA1.WPRE.hGHpA vector

SEQ ID NO: 19—pAAV.hSYN.hGBA1.WPRE.hGHpA vector

SEQ ID NO: 20—CAG promoter sequence operably linked to the GBA1 coding sequence

SEQ ID NO: 21—CAG promoter sequence operably linked to the GBA1 coding sequence and the WPRE sequence

### DETAILED DESCRIPTION OF THE INVENTION

It is to be understood that different applications of the disclosed polynucleotide sequences may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a polynucleotide” includes “polynucleotides”, reference to “a promoter” includes “promoters”, reference to “a vector” includes two or more such vectors, and the like.

All publications, patents and patent applications cited herein, whether supra or infra, are hereby incorporated by reference in their entirety.

The present invention concerns gene therapy for the treatment and/or prevention of Gaucher disease.

The present invention also concerns gene therapy for the treatment and/or prevention of diseases that are associated with the loss of GBA1 function, including other lysosomal storage disorders such as Niemann-Pick disease type C (NPC), and synucleinopathies including Parkinson's disease, dementia with Lewy bodies, multi-system atrophy (MSA) or pure autonomic failure (PAF).

The present invention concerns gene therapy for the treatment and/or prevention of lysosomal storage disorders or synucleinopathies. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human.

The expression constructs and vectors of the present invention can be used to treat Gaucher disease. Gaucher disease is a lysosomal storage disorder.

The expression constructs and vectors of the present invention can also be used to treat other lysosomal storage disorders or synucleinopathies.

Lysosomal Storage Disorders

Lysosomal storage disorders are monogenic metabolic diseases caused by the accumulation of biological materials in the late endosome/lysosome system. These include more than 60 different diseases, and even though they are referred to as rare their estimated combined frequency at birth is 1:7,500.

Lysosomal storage disorders include Sphingolipidoses such as Fabry disease, Farber lipogranulomatosis, Gaucher disease type I, Gaucher disease types II and III, Niemann-Pick disease types A and B, GM1-gangliosidosis: infantile, juvenile and adult variants, GM2-gangliosidosis (Sandhoff): infantile and juvenile, GM2-gangliosidosis (Tay-Sachs): infantile, juvenile and adult variants, GM2-gangliosidosis (GM2-activator deficiency), GM3-gangliosidosis, Metachromatic leukodystrophy (late infantile, juvenile and adult) and Sphingolipid-activator deficiency; Mucopolysaccharidoses such as MPS I (Scheie, Hurler-Scheie and Hurler disease), MPS II (Hunter), MPS IIIA (Sanfilippo A), MPS III (Sanfilippo B), MPS IIIC (Sanfilippo C), MPS IIID (Sanfilippo D), MPS IVA (Morquio syndrome A), MPS IVB (Morquio syndrome B), MPS VI (Maroteaux-Lamy), MPS VII (Sly disease) and MPS IX; Glycogen storage diseases such as Pompe (glycogen storage disease type II); Oligosaccharidoses such as α-Mannosidosis, β-Mannosidosis, Fucosidosis, Aspartylglucosaminuria, Schindler disease, Sialidosis, Galactosialidosis, Mucolipidosis II (I-cell disease) and mucolipidosis III; Integral membrane protein disorders such as Cystinosis, Danon disease, Action myoclonus-renal failure syndrome, Salla disease, Niemann-Pick disease type C1 and Mucolipidosis IV; and disorders such as Multiple sulphatase deficiency, Niemann-Pick disease type C2, Wolman disease (infantile), cholesteryl ester storage disease and Galactosialidosis.

Traditionally, lysosomal storage diseases have been classified according to the substrate that accumulates in the cells. However, these diseases are mainly caused by mutations in the genes encoding enzymatic hydrolases involved in the metabolism of macromolecules, so that the same metabolic pathway can be affected in different pathologies. Therefore, although caused by different genetic defects, distinctive disorders could be characterised by the accumulation of the same biological material. Moreover, the identification of novel defects in lysosomal enzymes and integral proteins involved in trafficking broadened the traditional classification of lysosomal storage disorders.

The pathophysiology of lysosomal storage disorders is complex. The endosome/lysosome system is a tightly connected cellular compartment and it is responsible for the degradation and recycling of extracellular substrates. Moreover, cellular components, such as protein aggregates, damaged cytosolic organelles and intracellular pathogens can be targeted for degradation in lysosomes through the formation of autophagosomes and consequent fusion and release of the damaged cellular material into the lysosomal compartment. Autophagy is a tightly controlled cellular mechanism; therefore it is not surprising that this process is dysregulated in many lysosomal storage disorders. Indications of the involvement of impaired autophagy in lysosomal storage disorders have been found in several animal models of Neuronal Ceroid Lipofuscinoses, Pompe disease and Niemann-Pick type C.

Some of the macromolecules that accumulate in lysosomal storage disorders are involved in various signal transduction pathways, such as glycosaminoglycans (GAGs) in mucopolysaccharidoses, heparan sulphate in Hurler disease or galactosylceramide in Krabbe disease. Metabolites produced from the degradation of GAGs have a similar structure to the bacterial endotoxin lipopolysaccharide. When this material accumulates it can activate the Toll like receptor 4 (TLR4), leading to the secretion of proinflammatory cytokines like TNF-α, and the proapoptotic signalling molecule ceramide. Ceramide synthesis is also upregulated in some ceroid lipofuscinosis, where the accumulation of CLN proteins activates the production of ceramide, contributing to the increased apoptosis. Impairment of the fibroblast growth factor 2 signalling cascade due to the accumulation of heparan sulphate in neuronal precursor cells in Hurler syndrome promotes neurodegeneration and cell death. The aforementioned examples are only a few of the critical signal transduction pathways that can be impaired in lysosomal storage diseases, where dysfunctions in lipid rafts and trafficking result in cell death. However, the apoptotic pathway is not the only signalling mechanism that can be altered in lysosomal storage disorders. For instance, in Krabbe disease the accumulation of galactosylceramide's derivative psychosine activates the T-cell associated gene 8 (TDAG8) receptor, inhibiting physiological cytokinesis with consequent formation of multinucleated ‘globoid’ cells.

Endoplasmic reticulum (ER) functionality is also impaired in some lysosomal storage disorders. In Gaucher disease the accumulation of glucosylceramide (GlcCer) within neurons causes supraphysiological release of calcium from the ER to the cytosol, inducing the activation of the calcium channel ryanodine receptor and consequent increased response of glutamate in affected neurons. On the contrary, in Sandhoff and Tay-Sach diseases the reuptake of calcium into the ER via the sarco/endoplasmic reticulum ATPase (SERCA) is inhibited. The depletion of calcium within the endoplasmic reticulum affects the correct folding of proteins. It has been reported that a continued unfolded protein response (UPR) is induced by the accumulation of storage material in GM1-gangliosidosis, causing apoptosis through the caspase-12 pathway. As a result of alteration of calcium homeostasis neurotoxicity is promoted, ultimately leading to neuronal loss.

Although the nature of the biologic material that accumulates in different lysosomal storage disorders varies, many of these pathologies share common clinical features. Typically, these disorders have multi-organ presentations. The onset of the phenotypes varies and even though lysosomal storage disorders are usually not congenital, in most acute cases the manifestations can be present at birth. In many diseases, like Gaucher, Niemann-Pick, MPSs, and other sphingolipidoses, one of the first pathological manifestations is hepatosplenomegaly, often already present at birth. Cardiomyopathies, including cardiomegaly, heart failure and deposition of glycogen in the heart valves are associated with many lysosomal storage disorders and can be present in newborns, like in infantile Pompe disease, or have a later onset as in several shpingolipidoses. Severe respiratory manifestations have been described in Pompe disease patients, where muscular hypotonicity causes reduction in lung volume; as well as in NPC-2 and Farber disease patients.

Haematological and endocrine manifestations are also typical of lysosomal storage disorders: anaemia and thrombocytopenia are haematological features characteristic of Gaucher disease, while osteopenia and enlargement of endocrine glands are present in other lysosomal storage disorders, especially in MPSs patients. As secondary manifestation of haematological disorders and organomegaly, many lysosomal storage disease patients, including MPS, GM1-gangliosidosis, NP-C, Gaucher and Farber disease, present with hydrops fetalis. Abnormal bone formation, joint contractures and swelling usually develop later in Gaucher, Farber, MPS and GM1-gangliosidosis patients, although bone disease has been occasionally described in neonates. Various cutaneous manifestations, such as ichtchyosis, skin lesions and an increase in body hair are typical of Gaucher, MPSs and Fabry disease. New born patients can also present dysmorphic features, as coarse facies, depressed or absent nasal septum and unusual facial appearances.

The central and peripheral nervous systems are affected in many forms of lysosomal storage diseases, causing a variety of symptoms, including neurocognitive impairment, movement disorders, seizures, optical manifestations and deafness, which usually lead to premature death. Even though many lysosomal storage disorders share common neurological phenotypes, the pathological mechanisms underlying neurodegeneration can be various. Clearly, the accumulation of different substrates and their effects on neurons depend on the specific cell type, morphology and distribution: in fact, the storage can be widespread throughout the brain or affect just a restricted more vulnerable cell population. In Fabry disease deposits of primary substrate globotriaosylceramide have been found in a limited number of neurons in specific brain regions, such as the brain stem, hypothalamus, amygdala, and in the spinal cord.

It has been shown that the accumulation of sphingolipids, such as ceramide and glucosylceramide play a significant role in the neuroinflammatory response in lysosomal storage diseases. Whether primary neuronal damage triggers the activation of glial cells or neurodegeneration and neuronal death are caused by metabolic dysfunctions within microglia has not yet been clarified. It is evident that chronic inflammation and neurodegeneration are tightly correlated; however the neuropathology in lysosomal storage disorders is not triggered exclusively by primary storage in neurons. For instance, in α-mannosidosis brains are affected by primary accumulation of mannose-rich oligosaccharides, although secondary storage of GM2 and GM3-gangliosides specifically in cortical pyramidal neurons is thought to affect neuronal integrity and contribute to the formation of ectopic dendrites and axonal spheroids. Although severe neuropathology can be already present at birth, in most patients the onset of the first neurological symptoms may range from late infancy to adulthood in less acute chronic cases. As for the visceral manifestations, neurological clinical expressions can vary accordingly to the severity of the mutations. The heterogeneity in symptoms and onset can lead to misdiagnosis or delayed diagnosis, in particular for subjects without family history. Therefore, the initial screening requires confirmation through biochemical and\or genetic analysis.

Enzyme replacement therapy is today's standard approved treatment for many lysosomal storage disorders, including Gaucher disease type I, Fabry disease, Pompe disease and some MPSs. The concept of cross-correction developed after the discovery that many lysosomal enzymes are targeted to the lysosomes via the mannose-6-phosphate (M6P) receptor pathway, and the same receptor is also present on the surface of the plasma membrane. According to this mechanism the addition of a M6P group to a recombinant enzyme allows the cellular uptake by nearby cells of administered or secreted enzyme and facilitates its transport to the lysosomes. Thus, the necessity of correcting every cell is overcome and low levels of intracellular enzymatic activity can be sufficient to restore the metabolic defect. It is obvious that the cross-correction principle is limited to soluble enzymes and it is not suitable for disorders involving transmembrane proteins.

Although enzyme replacement therapy is safe and usually well tolerated, it presents some disadvantages: patients are subjected to continuous and frequent infusions; the cost of repetitive administrations is significant; often combination therapies, like bone marrow transplantation are required; and more importantly the currently approved products do not show any efficacy in the treatment of central nervous system pathologies. In fact, the infused recombinant enzyme is not able to cross the blood-brain barrier, even when administered at high dose.

An alternative approach is to use a small molecule drug that reduces the synthesis of the accumulating pathogenic substrate. This is known as substrate reduction therapy. An approved substrate reduction therapy consists of the administration of the imino sugar N-butyldeoxynojirimycin (miglustat), a competitive inhibitor of ceramide glucosyltransferase that blocks the biosynthesis of glucosylceramide and glucosylceramide-derived glycosphingolipids.

Although miglustat was first commercialised for Gaucher disease type I, it also has potential for treatment of other lysosomal storage disorders, such as Niemann-Pick type C, Fabry disease, and GM1 and GM2-gangliosidose, where secondary accumulation of glucosylceramide-based glycosphingolipids occurs. Moreover, miglustat has shown the ability to cross the blood-brain barrier and therefore it can be used as a treatment for neurological manifestations. The main side effect of miglustat medication is the development of severe gastrointestinal symptoms and occasional peripheral neuropathy and tremor.

Most lysosomal storage disorders are associated with mutations in genes that influence protein conformation, folding and trafficking resulting in unstable and degradable enzymes. Pharmacological chaperones are molecules that, binding to the nascent polypeptides, promote protein stability and inhibit mis-folding and protein aggregation. Pharmacological chaperone therapy had first been proposed as a treatment for Fabry disease, where 1-deoxygalactonojirimycin (migalastat hydrochloride) binds to the active site of α-galactosidase A, increasing its activity. More recently, Orphan Drug designation was granted to Arimoclomol© (Orphazyme AsP) as a potential treatment for Niemann-Pick type C patients. Arimoclomol© is a co-inducer of the heat-shock response that induces the expression of molecular chaperones like Hsp70, and activates natural cellular repair pathways. The treatment has already shown beneficial effects in pre-clinical studies on animal models of amyotrophic lateral sclerosis, spinal bulbar muscular atrophy and retinitis pigmentosa. The on-going phase 2 study (NCT02612129) is currently investigating the efficacy and safety of the drug on NP-C subjects. Since not all mutations will be responsive to potential chaperone therapy and the effects of the treatment may not always be sufficient, researchers are investigating the possibility of chaperone therapy in combination with other treatments.

Bone marrow transplant for lysosomal storage diseases has been widely performed in the last two decades with the aim of engrafting donor cells of haemapoietic origin to correct enzyme deficiency in the host. Although this treatment results in amelioration of some disorders, such as Hurler disease, bone marrow transplant is not effective for acute neurodegenerative phenotypes. Although donor-derived cells can be found in the cerebrospinal fluid, the small percentage of corrected microglia cells is not always enough to provide robust enzyme expression and correct the most severe cases of neurodegeneration. The expression constructs and vectors of the present invention can be used in the prevention and/or treatment of lysosomal storage disorders. In one embodiment of the invention, the lysosomal storage disorder is Niemann-Pick disease type C (NPC). In another embodiment of the invention, the neurological complications of Niemann-Pick disease type C (NPC) are prevented or treated by use of the expression constructs and vectors of the present invention.

### Gaucher Disease

Gaucher disease is the most common lysosomal storage disorder, with an approximate prevalence of 1:100,000 and annual incidence in the general population of 1:60,000. The condition is defined as an autosomal recessive disease characterized by the inability of the defective lysosomal enzyme β-glucocerebrosidase to efficiently degrade its substrate glucosylceramide. The disease is caused by mutations in the GBA1 gene, which encodes for the lysosomal enzyme β-glucocerebrosidase, resulting in the accumulation of storage material in macrophages within visceral organs and in some cases the brain of affected patients.

The gene encoding β-glucocerebrosidase (GBA1; MIM #606463) is located on chromosome 1 (1q21). It consists of 11 exons and 10 introns spanning a total of 7.6 kb. The cDNA sequence for GBA1 is 1.6 kb long. The GBA1 gene is transcribed into different mRNAs, mainly deriving from alternative splicing events, alternative polyadenylation sites and transcription of the pseudogene. The levels of mRNA vary in different tissues, and they are not predictive of the enzymatic activity.

The spectrum of clinical manifestations of Gaucher disease is broad and there can be a lack of direct correlation between genotype and phenotype. However, Gaucher disease has historically been broadly classified into three distinct types according to the absence (type I) or presence and severity of central nervous system impairment (type II and type III). While there is a commercially available treatment that can ameliorate some of the systemic manifestations, the neurological manifestations of the disease still remain incurable.

Type I-Type I is the most frequent form of Gaucher disease and its manifestations do not involve central nervous system impairment. The physical presentation is characterised by a number of visceral symptoms. This includes severe splenomegaly: the size of the spleen can reach 1500-3000 cm³, compared to the average 50-200 cm³ in healthy adults. Hypersplenism is accompanied by massive distention of the abdomen and consequent pancytopenia. Anaemia, thrombocytopenia and leukopenia can be accompanied by coagulation defects. Although enlargement of the liver is common, it usually does not lead to empathic failure. Bone disease is present in 70-100% of type I patients: individuals often develop bone crises, pathologic fractures, arthritis, osteonecrosis of the joints and collapse of the vertebrae. Pulmonary involvement includes pulmonary hypertension and interstitial lung disease. Renal and cardiac complications are less common. Gaucher disease type I has a broad range of onset: first symptoms can appear in early childhood and worsen with time, or they can manifest in adult patients.
Type II-Type II is described as the acute neuronopathic form of Gaucher disease. The onset of the disease is in the neonatal period and death occurs by age two to four years. However, some cases of perinatal-lethal Gaucher disease associated with hepatosplenomegaly, skin abnormalities and intrauterine death have been reported. Gaucher disease type II presents the same visceral manifestation of type I, with significant hepatosplenomegaly and pulmonary involvement. Patients develop ichthyosis, ranging from mild skin peeling to the “colloid baby” phenotype. The earliest neurological symptoms are strabismus and horizontal gaze palsy, hypertonic posturing and retroflexion of the head. Soon after birth, patients manifest difficulties in swallowing, seizures and progressive epilepsy. Death usually occurs following apnoea and laryngospasm as a consequence of extensive paralysis.
Type II-Type III is the chronic neuronopathic form of Gaucher disease. Individuals can manifest the first symptoms already in early infancy, however the progression of the disease is slow and lifespan can be prolonged to adulthood. The visceral pathology is extended and present at birth, while the neurological signs can appear before two years of age or manifest later in life. In some cases of chronic neurologic Gaucher disease, dementia and ataxia can be observed in the latest stage of the pathology. The clinical course not always depends on age of onset and rate of progression. Furthermore, a subset of patients can manifest an intermediate phenotype between type II and III, characterised by a late onset and rapid progression of acute neurodegeneration.

Neuronopathic forms of Gaucher disease are characterised by perivascular and parenchymal accumulation of Gaucher cells, with pronounced neuronal loss, neuronal atrophy and necrosis. In particular, the acute infantile form is characterised by a severe, widespread and rapid neurodegeneration. Neuronal loss is prominent in cortex, hippocampus, hypothalamus, nuclei of the midbrain, cerebellum and brain stem. Affected brains also show extensive astrogliosis, microglia activation and non-specific grey matter gliosis.

The expression constructs and vectors of the present invention can be used in the treatment or prevention of Gaucher disease. The expression constructs and vectors of the present invention can be used in the treatment or prevention of Gaucher disease Type I, II and/or III. The expression constructs and vectors of the present invention can be used in the treatment or prevention of neuronopathic forms of Gaucher disease. The expression constructs and vectors of the present invention can be used to treat or prevent Gaucher disease by expressing GBA1 in the lungs and bones of patients with Gaucher disease.

Synucleinopathies

Synucleinopathies (also called α-Synucleinopathies) are neurodegenerative diseases characterised by the abnormal accumulation of aggregates of alpha-synuclein protein in neurons, nerve fibres or glial cells. There are three main types of synucleinopathy: Parkinson's disease (PD), dementia with Lewy bodies (DLB), and multiple system atrophy (MSA). Pure autonomic failure (PAF) is also considered to be a synucleinopathy. There is growing literature suggesting that loss of GBA1 function leads to the abnormal accumulation of alpha-synuclein protein in neurons, nerve fibres or glial cells, which in turn leads to synucleinopathies. The mechanistic links between glucocerebrosidase and α-synuclein are unclear, but there appears to be an inverse correlation between the levels of glucocerebrosidase and α-synuclein. Experimental evidence also supports a direct interaction between α-synuclein and glucocerebrosidase.

Recent studies have indicated that there is an increased frequency of mutations in the GBA1 gene among patients with Parkinson's disease. Mutations in the GBA1 gene are one of the most common genetic risk factors for Parkinson's that have been identified. Mutations in the GBA1 gene have also been reported as significant risk factors for Lewy body disorders, such as dementia with Lewy bodies. Studies have also shown that Gaucher-disease-causing GBA variants are significantly associated with MSA cerebellar subtype (MSA-C) patients.

The expression constructs and vectors of the present invention can be used in the treatment or prevention of synuclcinopathies. The expression constructs and vectors of the present invention can be used in the treatment or prevention of synucleinopathies such as Parkinson's disease, dementia with Lewy bodies, multiple system atrophy and pure autonomic failure (PAF).

### Expression Constructs of the Invention

An expression construct may be defined as a polynucleotide sequence capable of driving protein expression from a polynucleotide sequence containing a coding sequence. The expression constructs of the present invention comprise promoters and GBA1. The sequence of GBA1 used in the expression constructs of the present invention is preferably either that of SEQ ID NO: 1 or a GBA1 sequence encoding the polypeptide of SEQ ID NO: 12. The promoters used in the expression constructs of the present invention are the CBA or CAG promoters. The CBA (chicken beta-actin) promoter displays ubiquitous expression in cells. The sequence of the CBA promoter used in the present invention is preferably that of SEQ ID NO: 2. The CAG promoter is a ubiquitous expression promoter and is made up of the following elements: (C) the cytomegalovirus (CMV) early enhancer element, (A) the promoter, the first exon and the first intron of chicken beta-actin gene, (G) the splice acceptor of the rabbit beta-globin gene. The sequence of the CAG promoter used in the present invention is preferably that of SEQ ID NO: 3. A ubiquitous promoter can be defined as one which drives gene expression in a wide range of cells and tissues.

By using the ubiquitous CBA or CAG promoters in the expression constructs of the present invention, both systemic and neuronal expression of GBA1 can be achieved. By using the ubiquitous CBA or CAG promoters in the expression constructs of the invention, expression of GBA1 can be achieved in a wide range of tissues in patients with Gaucher disease, such as in the lungs and bones.

The CBA or CAG promoters for use in the present invention are operably linked to GBA1. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the expression construct.

An expression constructs of the present invention may also include additional nucleotide sequences not naturally found in the CBA or CAG promoter region or GBA1. An expression construct of the present invention may also include additional nucleotide sequences 5′ to the promoter sequence of CBA or CAG, 3′ to the promoter sequence of CBA or CAG but 5′ to GBA1, and/or 3′ to GBA1.

The expression constructs of the present invention can also be used in tandem with other regulatory elements such as one or more further promoters or enhancers or locus control regions (LCRs).

Thus, the expression constructs of the present invention may comprise, in a 5′ to 3′ direction: the CBA promoter sequence as shown in SEQ ID NO: 2, and, either the GBA1 sequence as shown in SEQ ID NO: 1 or a GBA1 sequence encoding the polypeptide of SEQ ID NO: 12

In a preferred embodiment the expression constructs of the present invention may comprise SEQ ID NO: 5.

The expression constructs of the present invention may comprise, in a 5′ to 3′ direction: the CAG promoter sequence as shown in SEQ ID NO: 3, and, either the GBA1 sequence as shown in SEQ ID NO: 1 or a GBA sequence encoding the polypeptide of SEQ ID NO: 12

In a preferred embodiment the expression constructs of the present invention may comprise SEQ ID NO: 6 or SEQ ID NO: 20.

In a preferred embodiment of the invention, the expression constructs of the present invention also comprise the WPRE (Woodchuck Hepatitis Virus (WHP) Posttranscriptional Regulatory Element) sequence. Studies have shown that, for example, a single-stranded AAV9 vector that includes a WPRE sequence has an enhanced expression profile in various organs when compared to a self-complementary vector configuration that lacks a WPRE sequence.

Thus, the expression constructs of the present invention may comprise, in a 5′ to 3′ direction: the CBA promoter sequence as shown in SEQ ID NO: 2, either the GBA1 sequence as shown in SEQ ID NO: 1 or a GBA sequence encoding the polypeptide of SEQ ID NO: 12, and the WPRE sequence as shown in SEQ ID NO: 4.

In a preferred embodiment the expression constructs of the present invention may comprise SEQ ID NO: 7.

The expression constructs of the present invention may comprise, in a 5′ to 3′ direction: the CAG promoter sequence as shown in SEQ ID NO: 3, and the GBA1 sequence as shown in SEQ ID NO: 1, and the WPRE sequence as shown in SEQ ID NO: 4.

In a preferred embodiment the expression constructs of the present invention may comprise SEQ ID NO: 8 or SEQ ID NO: 21.

Vectors of the invention may also incorporate codon-optimised sequences encoding a GBA1 polypeptide. These can be synthesised and incorporated into vectors of the invention using techniques described herein and/or known in the art. Exemplary codon-optimised sequences are shown in SEQ ID NO: 13-16.

In a preferred embodiment the expression constructs of the present invention may comprise the codon optimised GBA1 sequence of SEQ ID NO: 13.

In a preferred embodiment the expression constructs of the present invention may comprise the codon optimised GBA1 sequence of SEQ ID NO: 14.

In a preferred embodiment the expression constructs of the present invention may comprise the codon optimised GBA1 sequence of SEQ ID NO: 15.

In a preferred embodiment the expression constructs of the present invention may comprise the codon optimised GBA1 sequence of SEQ ID NO: 16.

Further expression constructs of the invention may comprise promoters that differ in sequence from the CBA or CAG promoter sequences above but retain the ability to express GBA1 in cells. Such sequences have at least 75%, at least 80%, at least 85%, at least 90%, at least 95%, at least 98% or at least 99% sequence identity to a sequence of contiguous nucleotides from SEQ ID NOs: 2 or 3.

Percentage sequence identity of variants is preferably measured over the full length of SEQ ID NO: 2, or over a 200, 210, 220, 230, 240 or 250 nucleotide section of SEQ ID NO: 2 aligned with the variant sequence.

Percentage sequence identity of variants is preferably measured over the full length of SEQ ID NOs: 3, or over a 550, 580, 600, 610, 620, 630 or 640 nucleotide section of SEQ ID NO: 3 aligned with the variant sequence.

Such variant sequences may preferably have at least 75%, at least 80%, at least 85%, at least 90%, at least 95%, at least 98% or at least 99% sequence identity to SEQ ID NOs: 2 or 3.

Retaining the ability to express GBA1 in cells can be measured by any suitable standard technique known to the person skilled in the art, for example, RNA expression levels can be measured by quantitative real-time PCR. Protein expression can be measured by western blotting or immunohistochemistry.

Further expression constructs of the invention comprise variants of GBA1 that retain the functionality of GBA1. A variant of GBA1 may be defined as any variant of the sequence of SEQ ID NO: 1, including naturally occurring variants in the nucleic acid sequence. The variant may be defined as having at least about 70%, 80%, 90%, 95%, 96%, 97%, 98% or 99% sequence identity to SEQ ID NO: 1, wherein the polypeptide translated from the variant sequence retains its functionality. Preferably, such variant sequences having at least 70%, 80%, 90%, 95%, 96%, 97%, 98% or 99% sequence identity to SEQ ID NO: 1 encode the polypeptide of SEQ ID NO: 12 or a polypeptide having at least 90%, 95%, 98% or 99% identity to SEQ ID NO: 12.

Further expression constructs of the invention comprise variants of GBA1 that encode the GBA1 polypeptide of SEQ ID NO: 12 and retain the functionality of GBA1. Such a variant may be any sequence encoding SEQ ID NO: 12, including naturally occurring variants in the nucleic acid sequence and optimised sequences such as those of SEQ ID NO: 13-16.

Other variants may be defined as sequences encoding a polypeptide having at least about 80%, 90%, 95%, 96%, 97%, 98% or 99% sequence identity to the sequence of SEQ ID NO: 12, wherein the polypeptide translated from the variant sequence retains its functionality.

Retaining GBA1 functionality can be defined as rescuing at least about 50%, 60%, 70%, 80% 90%, 95%, 96%, 97%, 98%, 99% or 100% of GBA1 function.

GBA1 function can be analysed by any suitable standard technique known to the person skilled in the art, for example, by a GBA enzymatic assay.

“Codon optimization” relates to the process of altering a naturally occurring polynucleotide sequence to enhance expression in the target organism, for example, humans. In one embodiment of the present invention, GBA1 is codon optimised. In some preferred embodiments, the codon optimised sequences of GBA1 are SEQ ID NOs: 13 to 16.

Further expression constructs of the invention may comprise WPRE sequences that differ in sequence from the WPRE sequence of SEQ ID NO: 4 but retain the functionality of the WPRE sequence. Such sequences have at least 75%, at least 80%, at least 85%, at least 90%, at least 95%, at least 98% or at least 99% sequence identity to a sequence of contiguous nucleotides from SEQ ID NO: 4.

Percentage sequence identity of variants is preferably measured over the full length of SEQ ID NO: 4, or over a 550, 560, 570, 580, 590 or 600 nucleotide section of SEQ ID NO: 4 aligned with the variant sequence. Such a variant sequence may preferably have at least 75%, at least 80%, at least 85%, at least 90%, at least 95%, at least 98% or at least 99% sequence identity to SEQ ID NOs: 4.

Retaining the functionality of the WPRE sequence can be defined as retaining at least about 50%, 60%, 70%, 80% 90%, 95%, 96%, 97%, 98%, 99% or 100% of the functionality of the WPRE sequence. WPRE function can be analysed by any suitable standard technique known to the person skilled in the art, for example, by monitoring RNA stability of the expression construct, by measuring RNA expression levels by quantitative real-time PCR, and/or by measuring protein expression of GBA1 by western blotting or immunohistochemistry.

Further expression constructs of the invention comprise variants of SEQ ID NO: 5; or SEQ ID NOs: 6 or 20 that differ in sequence from SEQ ID NO: 5; or SEQ ID NOs: 6 or 20 but retain the ability to express a functional form of GBA1 in cells. Such sequences have at least 75%, at least 80%, at least 85%, at least 90%, at least 95%, at least 98% or at least 99% sequence identity to SEQ ID NO: 5; or SEQ ID NOs: 6 or 20. GBA1 function can be analysed by any suitable standard technique known to the person skilled in the art, for example, by a GBA enzymatic assay. Preferably, the expression construct variants of SEQ ID NO: 5 described above comprise a region that has at least 90% sequence identity to SEQ ID NO: 2 that retains the ability to express GBA1, and/or (i) SEQ ID NO: 1 or (ii) a GBA1 sequence encoding the polypeptide of SEQ ID NO: 12 that retains the functionality of GBA1. The expression construct variants of SEQ ID NO: 5 described above encompass variants comprising a sequence that has at least 90% sequence identity to SEQ ID NO: 2 that retains the ability to express GBA1, and a sequence that has at least 90% sequence identity to (i) SEQ ID NO: 1 or (ii) a GBA1 sequence encoding the polypeptide of SEQ ID NO: 12 that retains the functionality of GBA1. Preferably, the expression construct variants of SEQ ID NOs: 6 or 20 described above comprise a region that has at least 90% sequence identity to SEQ ID NO: 3 that retains the ability to express GBA1, and/or SEQ ID NO: 1 or a GBA1 sequence encoding the polypeptide of SEQ ID NO: 12 that retains the functionality of GBA1. The expression construct variants of SEQ ID NOs: 6 or 20 described above encompass variants comprising a sequence that has at least 90% sequence identity to SEQ ID NO: 3 that retains the ability to express GBA1, and a sequence that has at least 90% sequence identity to (i) SEQ ID NO: 1 or (ii) a GBA1 sequence encoding the polypeptide of SEQ ID NO: 12 that retains the functionality of GBA1.

Further expression constructs of the invention comprise variants of SEQ ID NOs: 7 or 8 that differ in sequence from SEQ ID NOs: 7 or 8 but retain the ability to express a functional form of GBA1 in cells. Such sequences have at least 75%, at least 80%, at least 85%, at least 90%, at least 95%, at least 98% or at least 99% sequence identity to SEQ ID NOs 7 or 8. GBA1 function can be analysed by any suitable standard technique known to the person skilled in the art, for example, by a GBA enzymatic assay.

Preferably, the expression construct variants of SEQ ID NO: 7 described above comprise a region that has at least 90% sequence identity to SEQ ID NO: 2 that retains the ability to express GBA1, and/or (i) SEQ ID NO: 1 or (ii) a GBA1 sequence encoding the polypeptide of SEQ ID NO: 12 that retains the functionality of GBA1, and/or SEQ ID NO: 4 that retains the functionality of the WPRE sequence. The expression construct variants of SEQ ID NO: 7 described above encompass variants comprising a sequence that has at least 90% sequence identity to SEQ ID NO: 2 that retains the ability to express GBA1, a sequence that has at least 90% sequence identity to (i) SEQ ID NO: 1 or (ii) a GBA1 sequence encoding the polypeptide of SEQ ID NO: 12 that retains the functionality of GBA1, and a sequence that has at least 90% sequence identity to SEQ ID NO: 4 that retains the functionality of the WPRE sequence. Preferably, the expression construct variants of SEQ ID NO: 8 or 21 described above comprise a region that has at least 90% sequence identity to SEQ ID NO: 3 that retains the ability to express GBA1, and/or (i) SEQ ID NO: 1 or (ii) a GBA1 sequence encoding the polypeptide of SEQ ID NO: 12 that retains the functionality of GBA1, and/or SEQ ID NO: 4 that retains the functionality of the WPRE sequence. The expression construct variants of SEQ ID NO: 8 or 21 described above encompass variants comprising a sequence that has at least 90% sequence identity to SEQ ID NO: 3 that retains the ability to express GBA1, a sequence that has at least 90% sequence identity to (i) SEQ ID NO: 1 or (ii) a GBA1 sequence encoding the polypeptide of SEQ ID NO: 12 that retains the functionality of GBA1, and a sequence that has at least 90% sequence identity to SEQ ID NO: 4 that retains the functionality of the WPRE sequence.

Sequence identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

The expression constructs of the present invention can be used to drive significantly increased expression of GBA1 in cells. Significant increased expression can be defined as more than about 10 times, 20 times, 50 times, 100 times, 200 times or 300 times the expression of GBA1 in cells when compared with wild-type expression of GBA1. Expression of GBA1 can be measured by any suitable standard technique known to the person skilled in the art. For example, RNA expression levels can be measured by quantitative real-time PCR. Protein expression can be measured by western blotting or immunohistochemistry.

Vectors

The present invention provides vectors comprising the expression constructs of the present invention. The vector may be of any type, for example it may be a plasmid vector or a minicircle DNA.

Typically, vectors of the invention are however viral vectors. The viral vector may be based on the herpes simplex virus, adenovirus or lentivirus. The viral vector may be an adeno-associated virus (AAV) vector or a derivative thereof.

The viral vector derivative may be a chimeric, shuffled or capsid modified derivative.

The viral vector may comprise an AAV genome from a naturally derived serotype, isolate or clade of AAV.

The serotype may for example be AAV2, AAV5 or AAV8.

The efficacy of gene therapy is, in general, dependent upon adequate and efficient delivery of the donated DNA. This process is usually mediated by viral vectors. Adeno-associated viruses (AAV), a member of the parvovirus family, are commonly used in gene therapy. Wild-type AAV, containing viral genes, insert their genomic material into chromosome 19 of the host cell (Kotin, et al. 1990). The AAV single-stranded DNA genome comprises two inverted terminal repeats (ITRs) and two open reading frames, containing structural (cap) and packaging (rep) genes (Hermonat et al. 1984).

For therapeutic purposes, the only sequences required in cis, in addition to the therapeutic gene, are the ITRs. The AAV virus is therefore modified: the viral genes are removed from the genome, producing recombinant AAV (rAAV). This contains only the therapeutic gene, the two ITRs. The removal of the viral genes renders rAAV incapable of actively inserting its genome into the host cell DNA. Instead, the rAAV genomes fuse via the ITRs, forming circular, episomal structures, or insert into pre-existing chromosomal breaks. For viral production, the structural and packaging genes, now removed from the rAAV, are supplied in trans, in the form of a helper plasmid.

AAV is a particularly attractive vector as it is generally non-pathogenic; the majority people have been infected with this virus during their life with no adverse effects (Erles et al. 1999). Despite this, there are several drawbacks to the use of rAAV in gene therapy, although the majority of these only apply to systemic administration of rAAV. Nevertheless, it is important to acknowledge these potential limitations, even if not directly relevant to ocular administration of rAAV. Infection can trigger the following immunological responses:

As the majority of the human population is seropositive for AAV, neutralising antibodies against rAAV can impair gene delivery (Moskalenko et al. 2000; Sun et al. 2003).

Systemically delivered rAAV can trigger a capsid protein-directed T-cell response, leading to the apoptosis of transduced cells (Manno et al. 2006).

rAAV vectors can trigger complement activation (Zaiss et al. 2008).

As the rAAV delivery is generally unspecific, the vector can accumulate in the liver (Michelfelder et al. 2009).

AAV vectors are limited by a relatively small packaging capacity of roughly 4.8 kb and a slow onset of expression following transduction (Dong et al. 1996).

Most vector constructs are based on the AAV serotype 2 (AAV2). AAV2 binds to the target cells via the heparin sulphate proteoglycan receptor (Summerford and Samulski 1998). The AAV2 genome, like those of all AAV serotypes, can be enclosed in a number of different capsid proteins. AAV2 can be packaged in its natural AAV2 capsid (AAV2/2) or it can be pseudotyped with other capsids (e.g. AAV2 genome in AAV1 capsid; AAV2/1, AAV2 genome in AAV5 capsid; AAV2/5 and AAV2 genome in AAV8 capsid; AAV2/8).

rAAV transduces cells via serotype specific receptor-mediated endocytosis. A major factor influencing the kinetics of rAAV transgene expression is the rate of virus particle uncoating within the endosome (Thomas et al. 2004). This, in turn, depends upon the type of capsid enclosing the genetic material (Ibid.). After uncoating the linear single-stranded rAAV genome is stabilised by forming a double-stranded molecule via de novo synthesis of a complementary strand (Vincent-Lacaze et al. 1999). The use of self-complementary DNA may bypass this stage by producing double-stranded transgene DNA. Natkunarajah et al. found that self-complementary AAV2/8 gene expression was of faster onset and higher amplitude, compared to single-stranded AAV2/8 (2008). Thus, by circumventing the time lag associated with second-strand synthesis, gene expression levels are increased, when compared to transgene expression from standard single-stranded constructs. Subsequent studies investigating the effect of self-complementary DNA in other AAV pseudotypes (e.g. AAV2/5) have produced similar results (Kong et al. 2010; Petersen-Jones et al. 2009). One caveat to this technique is that, as AAV has a packaging capacity of approximately 4.8 kb, the self-complementary recombinant genome must be appropriately sized (i.e. 2.3 kb or less).

In addition to modifying packaging capacity, pseudotyping the AAV2 genome with other AAV capsids can alter cell specificity and the kinetics of transgene expression.

### AAV Genome

The vector of the present invention may comprise an adeno-associated virus (AAV) genome or a derivative thereof.

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly and with the additional removal of the AAV rep and cap genes, the AAV genome of the vector of the invention is replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype or isolate or clade of AAV. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, recently identified from primate brain. In vectors of the invention, the genome may be derived from any AAV serotype. The capsid may also be derived from any AAV serotype. The genome and the capsid may be derived from the same serotype or different serotypes.

In vectors of the invention, it is preferred that the genome is derived from AAV serotype 2 (AAV2), AAV serotype 4 (AAV4), AAV serotype 5 (AAV5) or AAV serotype 8 (AAV8). It is most preferred that the genome is derived from AAV2 but other serotypes of particular interest for use in the invention include AAV4, AAV5 and AAV8. It is preferred that the capsid is derived from AAV9.

In a preferred embodiment of the invention the genome is derived from AAV serotype 2 (AAV2) and the capsid is derived from AAV9, i.e. AAV2/9.

In a preferred embodiment of the present invention, the vector comprises the sequence of SEQ ID NO: 9 or SEQ ID NO: 17; or SEQ ID NO: 10 or SEQ ID NO: 18. The vectors of the present invention also encompass variants of SEQ ID NOs: 9 or 17; or SEQ ID NOs: 10 or 18 that differ in sequence from SEQ ID NOs: 9 or 17; or SEQ ID NOs: 10 or 18 but retain the ability to express a functional form of GBA1 in cells. Such sequences have at least 75%, at least 80%, at least 85%, at least 90%, at least 95%, at least 98% or at least 99% sequence identity to SEQ ID NOs: 9 or 17; or SEQ ID NOs: 10 or 18. GBA1 function can be analysed by any suitable standard technique known to the person skilled in the art, for example, by a GBA enzymatic assay. Preferably, the vector variants of SEQ ID NO: 9 or SEQ ID NO: 17 described above comprise a region that has at least 90% sequence identity to SEQ ID NO: 2 that retains the ability to express GBA1, and/or (i) SEQ ID NO: 1 or (ii) a GBA1 sequence encoding the polypeptide of SEQ ID NO: 12 that retains the functionality of GBA1, and/or SEQ ID NO: 4 that retains the functionality of the WPRE sequence. Preferably, the vector variants of SEQ ID NO: 10 or SEQ ID NO: 18 described above comprise a region that has at least 90% sequence identity to SEQ ID NO: 3 that retains the ability to express GBA1, and/or (i) SEQ ID NO: 1 or (ii) a GBA1 sequence encoding the polypeptide of SEQ ID NO: 12 that retains the functionality of GBA1, and/or SEQ ID NO: 4 that retains the functionality of the WPRE sequence.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may be used in the invention include:

Clade A: AAV1 NC_002077, AF063497, AAV6 NC_001862, Hu. 48 AY530611, Hu 43 AY530606, Hu 44 AY530607, Hu 46 AY530609

Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,

Clade C: Hu9 AY530629, Hu10 AY530576, Hu11 AY530577, Hu53 AY530615, Hu55 AY530617, Hu54 AY530616, Hu7 AY530628, Hu18 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623

Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013

Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu17 AY530582, Hu6 AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rh1 AY530556

Clade F: Hu14 (AAV9) AY530579, Hu31 AY530596, Hu32 AY530597, Clonal Isolate AAV5 Y18065, AF085716, AAV 3 NC_001729, AAV 3B NC_001863, AAV4 NC_001829, Rh34 AY243001, Rh33 AY243002, Rh32 AY243003/

The skilled person can select an appropriate serotype, clade, clone or isolate of AAV for use in the present invention on the basis of their common general knowledge.

It should be understood however that the invention also encompasses use of an AAV genome of other serotypes that may not yet have been identified or characterised. The AAV serotype determines the tissue specificity of infection (or tropism) of an AAV virus.

Typically, the AAV genome of a naturally derived serotype or isolate or clade of AAV comprises at least one inverted terminal repeat sequence (ITR). Vectors of the invention typically comprise two ITRs, preferably one at each end of the genome. An ITR sequence acts in cis to provide a functional origin of replication, and allows for integration and excision of the vector from the genome of a cell. Preferred ITR sequences are those of AAV2 and variants thereof. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (Virology Journal, 2007, 4:99), and in Choi et al and Wu et al, referenced above.

Derivatives of an AAV genome include any truncated or modified forms of an AAV genome which allow for expression of a Rep-1 transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs will preferably flank the expression construct cassette containing the promoter and transgene of the invention. The inclusion of one or more ITRs is preferred to aid packaging of the vector of the invention into viral particles. In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

With reference to the AAV2 genome, the following portions could therefore be removed in a derivative of the invention: One inverted terminal repeat (ITR) sequence, the replication (rep) and capsid (cap) genes. However, in some embodiments, including in vitro embodiments, derivatives may additionally include one or more rep and/or cap genes or other viral sequences of an AAV genome.

A derivative may be a chimeric, shuffled or capsid-modified derivative of one or more naturally occurring AAV viruses. The invention encompasses the provision of capsid protein sequences from different serotypes, clades, clones, or isolates of AAV within the same vector. The invention encompasses the packaging of the genome of one serotype into the capsid of another serotype i.e. pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population.

The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The vector of the invention takes the form of a viral vector comprising the expression constructs of the invention.

For the avoidance of doubt, the invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The invention additionally provides a host cell comprising a vector or AAV viral particle of the invention.

Preparation of Vector

The vector of the invention may be prepared by standard means known in the art for provision of vectors for gene therapy. Thus, well established public domain transfection, packaging and purification methods can be used to prepare a suitable vector preparation.

As discussed above, a vector of the invention may comprise the full genome of a naturally occurring AAV virus in addition to a promoter of the invention or a variant thereof. However, commonly a derivatised genome will be used, for instance a derivative which has at least one inverted terminal repeat sequence (ITR), but which may lack any AAV genes such as rep or cap.

In such embodiments, in order to provide for assembly of the derivatised genome into an AAV viral particle, additional genetic constructs providing AAV and/or helper virus functions will be provided in a host cell in combination with the derivatised genome. These additional constructs will typically contain genes encoding structural AAV capsid proteins i.e. cap, VP1, VP2, VP3, and genes encoding other functions required for the AAV life cycle, such as rep. The selection of structural capsid proteins provided on the additional construct will determine the serotype of the packaged viral vector.

A particularly preferred packaged viral vector for use in the invention comprises a derivatised genome of AAV2 in combination with AAV9 capsid proteins.

As mentioned above, AAV viruses are replication incompetent and so helper virus functions, preferably adenovirus helper functions will typically also be provided on one or more additional constructs to allow for AAV replication.

All of the above additional constructs may be provided as plasmids or other episomal elements in the host cell, or alternatively one or more constructs may be integrated into the genome of the host cell.

Expression constructs and vectors of the invention have the ability to rescue loss of GBA1 function, which may occur for example by mutations in the GBA1 gene. “Rescue” generally means any amelioration or slowing of progression of a Gaucher disease phenotype, for example restoring the presence of GBA1 protein in the visceral organs and the brain, thus visceral an neuronal pathologies.

The properties of the expression constructs and vectors of the invention can also be tested using techniques known by the person skilled in the art. In particular, a sequence of the invention can be assembled into a vector of the invention and delivered to a GBA1-deficient test animal, such as a mouse, and the effects observed and compared to a control.

### Methods of Therapy and Medical Uses

The expression constructs and vectors of the invention may be used in the treatment or prevention of Gaucher disease.

The expression constructs and vectors of the present invention can also be used in the treatment and/or prevention of diseases that are associated with that loss of GBA1 function, including other lysosomal storage disorders such as Niemann-Pick disease type C (NPC), and synucleinopathies including Parkinson's disease, dementia with Lewy bodies, multi-system atrophy (MSA) or pure autonomic failure (PAF).

The expression constructs and vectors of the invention may be used in the treatment or prevention of lysosomal storage disorders such as Niemann-Pick disease type C (NPC).

The expression constructs and vectors of the invention may be used in the treatment or prevention of synucleinopathies, such as Parkinson's disease, dementia with Lewy bodies, multiple system atrophy and pure autonomic failure (PAF).

This provides a means whereby the degenerative process of the diseases can be treated, arrested, palliated or prevented.

The invention therefore provides a pharmaceutical composition comprising the vector of the invention and a pharmaceutically acceptable carrier.

The invention also provides a vector for use in a method of preventing or treating Gaucher disease.

The invention also provides a vector for use in a method of preventing or treating other lysosomal storage disorders such as Niemann-Pick disease type C (NPC).

The invention also provides a vector for use in a method of preventing or treating synucleinopathies, such as Parkinson's disease, dementia with Lewy bodies, multiple system atrophy and pure autonomic failure (PAF).

The invention also provides the use of a vector of the invention in the manufacture of a medicament for the treatment or prevention of Gaucher disease.

The invention also provides the use of a vector of the invention in the manufacture of a medicament for the treatment or prevention of other lysosomal storage disorders such as Niemann-Pick disease type C (NPC).

The invention also provides the use of a vector of the invention in the manufacture of a medicament for the treatment or prevention of synucleinopathies, such as Parkinson's disease, dementia with Lewy bodies, multiple system atrophy and pure autonomic failure (PAF).

The invention also provides a method of treating or preventing Gaucher disease in a patient in need thereof comprising administering a therapeutically effective amount of a vector of the invention to the patient. The invention also provides a method of treating or preventing Gaucher disease in a patient in need thereof wherein the Gaucher disease is Type I, II or III. The invention also provides a method of treating or preventing Gaucher disease in a patient in need thereof wherein the Gaucher disease is neuronopathic.

By using the ubiquitous CBA or CAG promoters in the expression constructs of the invention, expression of GBA1 can be achieved in a wide range of tissues in patients with Gaucher disease, such as in the lungs and bones. Thus the expression constructs and vectors of the present invention can be used to treat or prevent Gaucher disease by expressing GBA1 in the lungs and/or bones of patients with Gaucher disease, and thus treating the lungs and/or bones of such patients.

The invention also provides a method of treating or preventing lysosomal storage disorders such as Niemann-Pick disease type C (NPC) in a patient in need thereof comprising administering a therapeutically effective amount of a vector of the invention to the patient. The invention also provides a method of treating or preventing synucleinopathies, such as Parkinson's disease, dementia with Lewy bodies, multiple system atrophy and pure autonomic failure (PAF) in a patient in need thereof comprising administering a therapeutically effective amount of a vector of the invention to the patient. In a preferred embodiment of the invention, the neurological complications of Niemann-Pick disease type C (NPC) are prevented or treated by use of the expression constructs and vectors of the present invention.

In general, parenteral routes of delivery of vectors of the invention, such as intravenous (IV) or intracerebroventricular (ICV) administration, typically by injection, are preferred.

The invention therefore also provides a method of treating or preventing Gaucher disease in a patient in need thereof, comprising administering a therapeutically effective amount of a vector of the invention to the patient by a parenteral route of administration. Accordingly, Gaucher disease is thereby treated or prevented in said patient.

The invention therefore also provides a method of treating or preventing other lysosomal storage disorders such as Niemann-Pick disease type C (NPC) in a patient in need thereof, comprising administering a therapeutically effective amount of a vector of the invention to the patient by a parenteral route of administration. Accordingly, lysosomal storage disorders such as Niemann-Pick disease type C (NPC) are thereby treated or prevented in said patient.

The invention therefore also provides a method of treating or preventing synucleinopathies, such as Parkinson's disease, dementia with Lewy bodies, multiple system atrophy and pure autonomic failure (PAF) in a patient in need thereof, comprising administering a therapeutically effective amount of a vector of the invention to the patient by a parenteral route of administration. Accordingly, synucleinopathies, such as Parkinson's disease, dementia with Lewy bodies, multiple system atrophy and pure autonomic failure (PAF) are thereby treated or prevented in said patient.

In a related aspect, the invention provides for use of a vector of the invention in a method of treating or preventing Gaucher disease by administering said vector to a patient by a parenteral route of administration. Additionally, the invention provides the use of a vector of the invention in the manufacture of a medicament for treating or preventing Gaucher disease by a parenteral route of administration.

The invention also provides for use of a vector of the invention in a method of treating or preventing other lysosomal storage disorders such as Niemann-Pick disease type C (NPC by administering said vector to a patient by a parenteral route of administration. Additionally, the invention provides the use of a vector of the invention in the manufacture of a medicament for treating or preventing lysosomal storage disorders such as Niemann-Pick disease type C (NPC) by a parenteral route of administration.

The invention provides for use of a vector of the invention in a method of treating or preventing synucleinopathies, such as Parkinson's disease, dementia with Lewy bodies, multiple system atrophy and pure autonomic failure (PAF) by administering said vector to a patient by a parenteral route of administration. Additionally, the invention provides the use of a vector of the invention in the manufacture of a medicament for treating or preventing synucleinopathies, such as Parkinson's disease, dementia with Lewy bodies, multiple system atrophy and pure autonomic failure (PAF) by a parenteral route of administration.

In all these embodiments, the vector of the invention may be administered in order to prevent the onset of one or more symptoms of Gaucher disease. The patient may be asymptomatic. The subject may have a predisposition to the disease. The method or use may comprise a step of identifying whether or not a subject is at risk of developing, or has, Gaucher disease.

In all these embodiments, the vector of the invention may be administered in order to prevent the onset of one or more symptoms of other lysosomal storage disorders such as Niemann-Pick disease type C (NPC), or synucleinopathies, such as Parkinson's disease, dementia with Lewy bodies, multiple system atrophy and pure autonomic failure (PAF). The patient may be asymptomatic. The subject may have a predisposition to the disease. The method or use may comprise a step of identifying whether or not a subject is at risk of developing, or has lysosomal storage disorders such as Niemann-Pick disease type C (NPC), or synucleinopathies, such as Parkinson's disease, dementia with Lewy bodies, multiple system atrophy and pure autonomic failure (PAF).

A prophylactically effective amount of the vector is administered to such a subject. A prophylactically effective amount is an amount which prevents the onset of one or more symptoms of the disease.

Alternatively, the vector may be administered once the symptoms of the disease have appeared in a subject i.e. to cure existing symptoms of the disease. A therapeutically effective amount of the antagonist is administered to such a subject. A therapeutically effective amount is an amount which is effective to ameliorate one or more symptoms of the disease.

The subject may be male or female. The subject is preferably identified as being at risk of, or having, the disease.

The administration of the vector is typically by a parenteral route of administration. Parenteral routes of administration encompass intravenous (IV), intramuscular (IM), subcutaneous (SC), epidural (E), intracerebral (IC), intracerebroventricular (ICV) and intradermal (ID) administration.

The dose of a vector of the invention may be determined according to various parameters, especially according to the age, weight and condition of the patient to be treated; the route of administration; and the required regimen. Again, a physician will be able to determine the required route of administration and dosage for any particular patient. For example, a suitable dose of a vector of the present invention may be in the range of 6.7×10¹³ vg/kg to 2.0×10¹⁴ vg/kg, where vg=viral genome.

The dose may be provided as a single dose, but may be repeated in cases where vector may not have targeted the correct region. The treatment is preferably a single permanent, but repeat injections, for example in future years and/or with different AAV serotypes may be considered.

### Host Cells

Any suitable host cell can be used to produce the vectors of the invention. In general, such cells will be transfected mammalian cells but other cell types, e.g. insect cells, can also be used. In terms of mammalian cell production systems, HEK293 and HEK293T are preferred for AAV vectors. BHK or CHO cells may also be used.

### Pharmaceutical Compositions and Dosages

The vector of the invention can be formulated into pharmaceutical compositions. These compositions may comprise, in addition to the vector, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Hartmann's solution. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

Dosages and dosage regimes can be determined within the normal skill of the medical practitioner responsible for administration of the composition.

### Combination Therapies

The expression constructs, vectors and/or pharmaceutical compositions can be used in combination with any other therapy for the treatment or prevention of Gaucher disease, such as enzyme replacement therapy (ERT) or substrate replacement therapy (SRT).

The expression constructs, vectors and/or pharmaceutical compositions can also be used in combination with any other therapy for the treatment or prevention of other lysosomal storage disorders such as Niemann-Pick disease type C (NPC) or synucleinopathies, such as Parkinson's disease, dementia with Lewy bodies, multiple system atrophy and pure autonomic failure (PAF), such as enzyme replacement therapy (ERT) or substrate replacement therapy (SRT).

### Kits

The expression constructs, vectors and/or pharmaceutical compositions can be packaged into a kit.

### EXAMPLES

### Materials and Methods

Creation of pAAV.CAG.GBA.WPRE and pAAV.CBA.GBA.WPRE Vectors

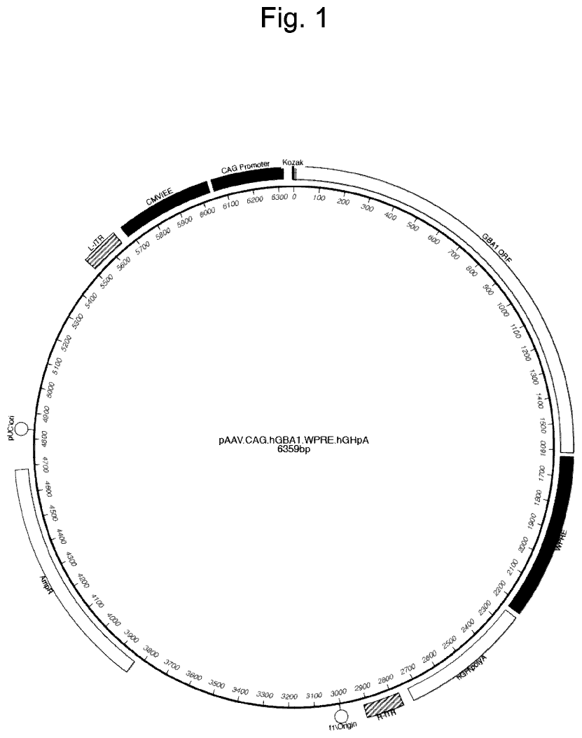

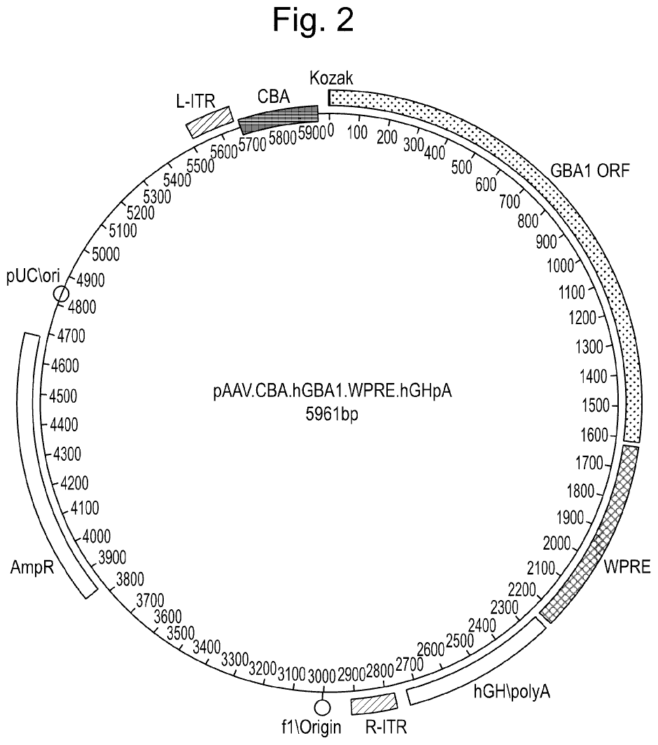

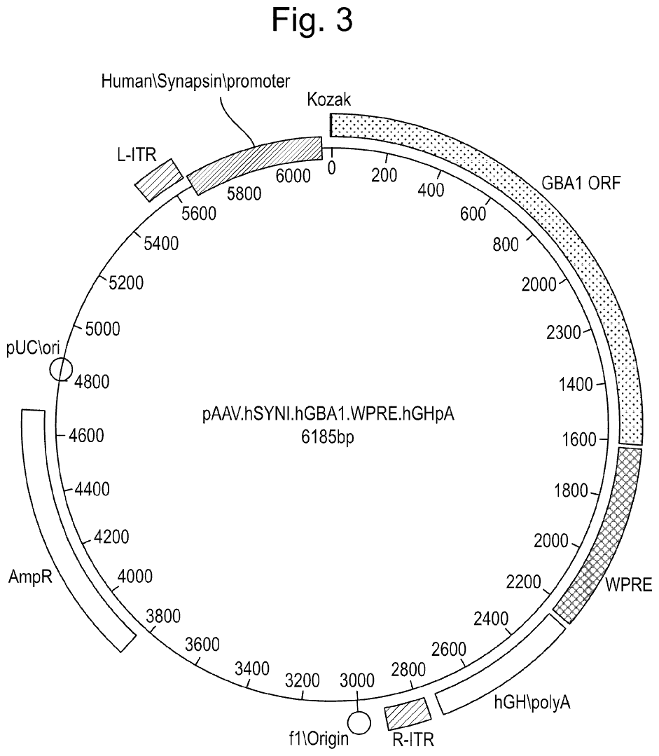

The pAAV.CBA.GBA.WPRE and pAAV.CAG.GBA.WPRE vectors were created using standard molecular biology protocols. For the CBA constructs, a pUC18.CBA vector from Genscript was used. For the CAG constructs, a pUC18.CAG vector from Genscript was used. After transformation of Stbl3 competent E. coli (ThermoFisher) and subsequent extraction of DNA, the pUC18.CBA and pUC18.CAG vectors were digested with MluI and BspEI (NEB), along with a pAAV9.hSYN.GBA1.WPRE.hGHpA vector (SEQ ID NO: 11 or SEQ ID NO:19 and FIG. 3). Thus the synapsin (SYN) promoter was removed and the CBA or CAG promoters ligated into the AAV9 vector. The sequences of pAAV.CBA.GBA.WPRE.hGHpA are set out in SEQ ID NO: 9 or SEQ ID NO: 17. A diagrammatic representation of pAAV.CBA.GBA.WPRE.hGHpA is provided in FIG. 2. The sequences of pAAV.CAG.GBA.WPRE.hGHpA are set out in SEQ ID NO:10 or SEQ ID NO: 18. A diagrammatic representation of pAAV.CAG.GBA.WPRE.hGHpA is provided in FIG. 1.

### Cloning

pAAV.hSynI.GBA. WPRE.hGHpA

The human GBA cDNA was amplified via PCR, introducing two restriction site sequences corresponding to the BspEI enzyme at the 5′ of the sequence and EcoRI at the 3′. The GBA sequence was then cloned into an expression cassette flanked by ssAAV2 inverted terminal repeats and containing the human synapsin promoter hSynI, the enhancing WPRE sequence and the human growth hormone polyadenilation signal sequence through restriction enzyme digest (New England Biolab) followed by ligation reaction (Promega). Plasmid sequences were confirmed via Automated Sanger sequencing (SourceBioscience).

pAAV.CBA.GBA.WPRE.hGHpA and pAAV.CAG.GBA.WPRE.hGHpA

The synapsin promoter was removed from the original pAAV.hSynI.GBA.WPRE.hGHpA plasmid through restriction enzyme digest with the MluI and BspEI enzymes at the 5′ and 3′ of the sequence respectively. Two sites corresponding to the same restriction enzymes were added to the 5′ and 3′ of the chicken-β-actin (CBA) and enhanced chicken-β-actin (CAG) promoter sequences (GenScript). CBA and CAG promoters were subsequently cloned into the original plasmid upstream the GBA cDNA via ligation reaction. Plasmid sequences were confirmed via Automated Sanger sequencing (SourceBioscience).

### Virus Production

Recombinant AAV9 serotype vectors encoding GBA (ssAAV9.SYN.GBA; ssAAV9.CAG.GBA; ssAAV9.CBA.GBA) were generated by the standard triple plasmid transfection method as described previously in Ayuso E. et al (2010) Gene Ther. 17: 503-510. Briefly, cell lysates of transfected 293 cells were clarified by centrifugation and then purified by subjecting the preparations to two cycles of density gradient ultracentrifugation using cesium chloride. The purified recombinant vectors were resuspended and stored in 10 mM sodium phosphate buffer, pH 7.3, containing 180 mM sodium chloride and 0.001% pluronic F68.

### Codon-Optimised Sequences

Codon-optimised GBA1 coding sequences encoding GBA1 polypeptides can also be used. Some exemplary codon-optimised sequences are provided in SEQ ID NO: 13-16. Codon-optimised sequences such as those of SEQ ID NO: 13-16 can be synthesised and incorporated into viral vectors using similar techniques to those described above for pAAV.CAG.GBA.WPRE and pAAV.CBA.GBA.WPRE.

### Virus Administration

At postnatal day 0-1 pups were anesthetised on ice for 30-60 seconds and intravenous injections were performed via the superficial temporal vein with 40 μl of vector using a 33-gauge needle (Hamilton) (Gombash Lampe et al., 2014). Once the needle was slowly removed, gentle pressure was applied to the injection site. When the pup fully recovered it was returned to the dam.

The bilateral intracerebroventricular injections were directed to the anterior horn of the lateral ventricle. The injection site was identified at ⅖ of the distance from the lambda suture to each eye (Kim et al., 2014). PO-1 mice were anesthetised on ice for 30-60 seconds. The needle was inserted perpendicularly at the injection site to a depth of 3 mm and 5 μl of vector was slowly administered. Following a brief pause to allow vector distribution, the contralateral ventricle was injected with the same volume of vector. The pup was allowed to recover and placed back into the cage.

Adult mice (P30) were injected into the lateral tail vein using a 33-gauge needle (Hamilton) (Walter et al., 1996). Animals were administered with 40 μl of vector.

### Animals

K14-Cre gbaInl/Inl knock-out mice (K14-Inl/Inl) were used as a model of acute neuronopatic Gaucher Disease (Enquist et al., 2007). Heterozygote mice (K14-Inl/wt) were mated to generate knock-out, heterozygotes and wild-type (K14 wt/wt) animals which were used as controls.

Animals were maintained on a 12 h light/dark cycle, with free access to water and food. Animals were group-housed into individually ventilated cages (IVCs) with appropriate litter and nesting material, and environmental enrichment elements. No more than two females aged from 6 to 15 weeks per stud male were weekly timed mated.

Mice were weighed weekly and were sacrificed if a loss of more than 15% of the total body mass was observed as a comprehensive humane endpoint. The animals were routinely monitored and culled if the humane endpoint was reached (mouse presents with paralysis, spasticity, neck hypertension or unconsciousness for more than 4 hours).

### Tattooing

Animals were identified through permanent paw tattooing performed at day of birth (Castelhano-Carlos et al., 2010). 2 μl of tattoo ink (Harvard Apparatus, Holliston Mass., USA. Dilution 1:3 in PBS) were injected subcutaneously into pups' palms using a 33-gauge needle (Hamilton, Cole-Parmer, London, UK). A 4-feet numbering system is used to identify different animals when house-grouped.

### Genotyping

Blood samples were taken from the temporal vein of PO mice. The vein was punctured with a 25 g needle (BD Microlance) and a drop of blood is collected with a pipette. A maximum of 10% of the total blood volume was collected. 10 μl of blood was blotted on filter paper (Whatman 903 paper, CDC 5-spot card, 100/pk, GE Healthcare, USA). The GCase enzymatic activity in blood samples was measured following incubation with the synthetic substrate 4-methylumbelliferyl-β-D-glucopyranoside.

### Behavioural Assessment

Animals were moved to the test room 20 minutes before the assessment. All tests are filmed and results recorded.

Open field test: The mouse was placed in the center of a square transparent Plexiglas chamber measuring 27 cm×27 cm and allowed to freely explore the chamber for 5 minutes (Bailey, 2009). Animals were then filmed from the top of the chamber for additional 5 minutes. The analysis of the tests was carried out using ANY-maze Behaviour Tracking Software v. 4.99 (Stoelting, Dublin, Ireland), assessing distance, average speed, mobility and immobility time of each animal.

### Sacrificing

2-month-old mice were euthanised by transcardial perfusion using PBS while under terminal isofluorane anesthesia.

### GCase Enzyme Assay

GCase activity was determined using the established synthetic substrate, 4-methylumbelliferone-β-glucopyranoside protocol as previously described in Wenger D. A. et al (1978) Clin Genet. 13:145-153. Frozen tissue samples were homogenized with distilled water on ice and the total protein concentration was measured using BCA assay. Samples were incubated with the substrate for 2 h at 37° C. The reaction was stopped with 1 M glycine buffer, pH 10.4. Fluorescence of the standard and the samples was read (Spectra Max M2, SoftMax Pro 4.6; excitation wavelength: 365 nm, emission wavelength: 450 nm). The enzymatic activity (nmol h⁻¹ μg⁻¹) was calculated.

### LC/MS-MS Substrate Measurement

Distilled water was added to tissues in a 3:1 ratio and homogenisation was carried out following the manufacturer's guidelines. 10 μl of homogenate was analyzed for glucosylceramide accumulation. The substrates were extracted in 10 volumes of methanol containing 25 ng ml-1 of 15-Hydroxyicosatetraenoic acid (15-HETE) obtained from Sigma Aldrich. The samples were shaken for 5 min at room temperature on a Bioshake at 2000 rpm and transferred to the −20° C. freezer for a minimum of 2 hours. After a 20 min centrifugation at 2,500 g, the supernatents were transferred to a 96-well plate and 1 μl injected into the ultra-high performance liquid chromatography-tandem mass spectrometry system. Glycosphingolipid reference standards (Matreya) were also analyzed to confirm analyte identity.

The samples were injected onto a Thermo Vanquish UHPLC system operated in partial loop mode and separated on a Phenomenex Luna Omega Polar C18 column (100 Å, 1.6 μm, 2.1 mm×50 mm) under the following gradient conditions: Initial 80% A, 0.00-0.70→0% A; 0.70-1.30→0% A; 1.30-1.40→80% A; 1.4-1.70→80% A, where mobile phase A was Milli-Q H₂O with 0.1% FA; phase B was isopropanol/acetonitrile (1:1 v/v) with 0.1% FA and the flow rate was 0.8 ml min⁻¹. Column and sample temperatures were kept at 65° C. and 6° C., respectively. Wash solvent was methanol/acetonitrile/isopropanol/H₂O (2:1:1:1 v/v). The eluting analytes were detected on a Thermo TSQ Quantiva triple quadrupole mass spectrometer that was equipped with the electrospray ion source and operated in multiple reaction monitoring and negative ion mode (see Supplementary Table 4 for multiple reaction monitoring details) with the tune page parameters set to achieve the maximum sensitivity for glycosphingolipids as described previously. The data were processed with Xcalibur v4.1.

### Immunohistochemical Staining

All tissues were fixed in 4% paraformaldehyde (PFA; Sigma-Aldrich) for 48 hr, transferred to a cryopreserving solution of 30% sucrose (w/v) (Sigma-Aldrich) in PBS and stored at 4° C. Fixed organs were embedded with specimen matrix (Thermo Scientific), frozen and cut at 40 μm in thickness at constant temperature of −20° C. with a Cryostat Leica CM3050 (Leica Biosystems, Milton Keynes, UK). Coronal brain sections were sliced starting from the front of the olfactory bulbs to the cerebellum and brain stem. Slices were stored at 4° C. in Tris Buffered Saline with anti-freeze (TBSAF) in 96-well plates. A series of representative sections were collected in six-well plates and washed three times with 3 ml of TBS with 5 minutes between each wash on a rocking table. Endogenous peroxidase activity was blocked with 1% H2O2 in 1×TBS for 30-60 minutes under constant gentle agitation. Slices were rinsed three times in 1×TBS and the non-specific binding was blocked in 15% normal serum (Sigma-Aldrich) in 1×TBS-T for 30 minutes on a rocking table. Sections were washed three times with 1×TBS and the primary antibody diluted with 10% normal serum in TBS-T was added. The plate was incubated at 4° C. overnight on a rocking table. The following day, slices were washed three times with 1×TBS and incubated for 2 hours at room temperature with the secondary antibody diluted with 10% normal serum in TBS-T. Afterwards, sections were rinsed three times in 1×TBS and incubated for 2 hours with 1:1000 avidine-biotin reagent (Vectastain Elite ABC kit, Vector Labs, UK). Sections were then washed three times in 1×TBS and immunoreaction was detected by adding 0.45 μm filtered 0.05% 3,3′-diaminobenzidine tetrahydrochloride (Sigma-Aldrich) and 0.001% H2O2 in 1×TBS (one DAB tablet in 20 ml of TBS). The plate was covered with foil and kept under agitation for a few minutes. The reaction was stopped by adding ice-cold 1×TBS. After three washes in 1×TBS, sections were mounted on chrome-gelatine coated slides and left to air-dry overnight. Slides were dehydrated in 100% ethanol, cleared in Histo-clear (National Diagnostic, Atlanta Ga., USA) for 30 minutes and cover slipped with DPX mountant (Fisher Scientific).

### Neuronal Counts and Cortical Thickness Measurements

Neuron counts and cortical thickness measurements were estimated with Stereo Investigator software (MBF Bioscience, Williston VE, USA) on Nissl stained sections with a Nikon Optihot light microscope (Nikon) attached to a Q-Imagin camera (MBF 2000R-CLR-12, Bioscience). The user was blinded to the experimental slides being analysed by another person covering the slide labels with tape.

Neurons were counted with the Optical Fractionator probe using the 40× objective. Efficient sampling was estimated by a coefficient of error between 0.05 and 0.1. 3 to 4 sections for each brain were analysed and the average values of cell counting were used in the calculations. The mean thickness of the S1BF cortical region was estimated by using the Cavalieri vertical sections principle. 3 sections of the midbrain region per each brain were analysed and the average values were reported.

### Determining Vector Copy Number

Quantitative PCR (qPCR) analysis of the WPRE region was used to examine the viral copy number following injection with SYN.GBA1, CAG.GBA1 and CBA.GBA1 at both doses in brain, liver, lung, spleen and heart tissue.

DNA was extracted from the sample homogenates using the DNeasy Blood & Tissue Kit (QIAGEN). Mouse TITIN was used as a housekeeping gene. The standard curves for WPRE and TITIN were constructed from plasmid stocks. Plasmid stocks were diluted to 1×10¹⁰ copies/μl in DEPC-treated, nuclease-free water (Fisher) then 10-fold serial dilutions in DEPC-treated, nuclease-free water was prepared. 1×10⁸ copies/μl was used for the top concentration of the standard curve for each template with the seven 10-fold serial dilutions. Per reaction, a master mix for each template was prepared that included iTaq Universal SYBR green supermix (BioRad 1725124) mixed with forward and reverse primers with DEPC-treated, nuclease-free water. 8 μl of the master mix was added per sample, qPCR was performed on an Agilent AriaMX Real-time machine.

For the melt curves for both TITIN and WPRE, a single peak was observed. A standard curve was constructed, with the logarithm of the initial copy number of the standards plotted along the x-axis and their respective C_{T} values plotted along the y-axis. Based on the equation for the linear regression for the standard curve, the equation: copies/μl=10^{(CT-b)/m} was used to determine the quantity of test sample (Biorad Real-time PCR applications guide). The final VCN was expressed as vector genome/diploid genomic equivalent (vg/gde).

### Example 1: Transfection of HEK Cells with GBA Plasmids

HEK293T PRO cells were transfected with the plasmids pAAV.CAG.GBA.WPRE, pAAV.CBA.GBA.WPRE or pAAV.SYN.GBA.WPRE. Untransfected cells were used as a control. Three wells of cells were transfected for each experimental plasmid (1 μg DNA per well). Three untransfected wells acted as the negative control.

48 hours post transfection, supernatants were collected from experimental and untransduced samples. Cells were collected in extraction buffer to extract and preserve the integrity of the β-glucocerebrosidase enzyme. A BSA assay performed using Pierce BCA protein assay kit, which showed that samples did not need to be diluted. Samples were stored at −20° C.

A GBA enzymatic assay was carried out on the collected supernatants and cells. Samples were performed in duplicate. Samples were incubated with the substrate (4-methylumbelliferyl-β-D-glucopyranoside) for 1 hour. Samples were measured against a standard (4-methylumbelliferone) and a standard blank (H₂O) for each sample. A Stop solution of 1M glycine buffer pH 10.4 was used to end the reaction. Fluorescence of the standard 1 nM 4-methylumbelliferone and the samples was read (FluoStar Optima Plate Reader. Excitation wavelength: 360 nm; emission wavelength: 450 nm). The enzymatic activity (nmol/hr/p) was calculated.

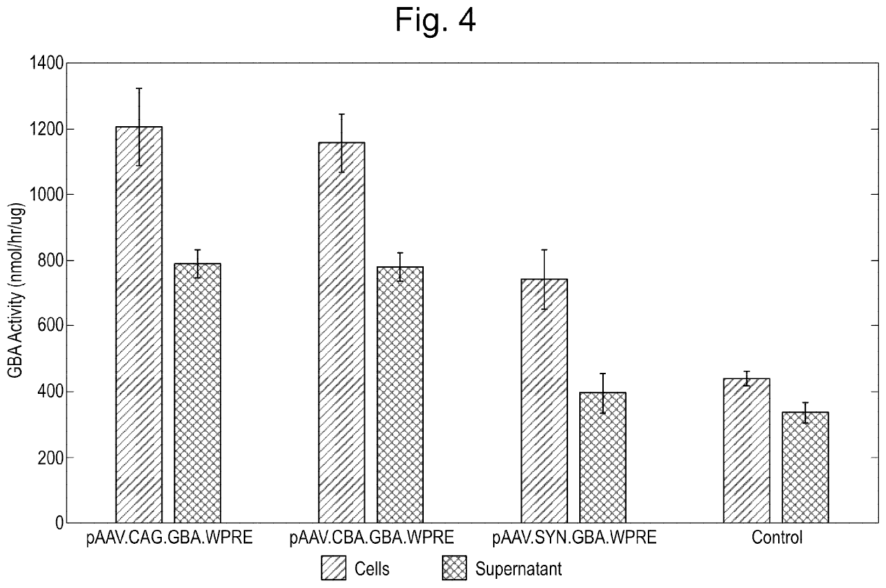

All transfected cells displayed significantly increased GBA activity compared to the untransduced control (FIG. 4). Synapsin gave lower GBA activity than CAG and CBA. However, this may be due to synapsin being a neuron-specific instead of ubiquitous promoter, and thus may not be greatly expressed in the kidney cell line used.

### Advantages of the Vectors of the Present Invention Over Gene Therapies Disclosed in the Art

The synapsin promoter used in disclosed Gaucher disease gene therapy vectors is a neuronal-selective promoter. The promoter targets neurons well and leads to high expression of GBA1 in neurons in the brain/CNS. Following IV injection of AAV SYN GBA1, there was some amelioration of visceral symptoms which would suggest that the gene was also expressed in non-neuronal cells to some extent. However, the expression in non-neuronal cells was limited to the liver and spleen. There was not a robust expression of GCase in the heart or lung.

The vectors of the present invention will be expressed in neuronal and non-neuronal cells, showing better expression of GCase in the heart and lung than the synapsin promoter.

The GusB promoter used in disclosed Gaucher disease gene therapy vectors drives expression in both neuronal and non-neuronal cells, therefore is referred to as a ubiquitous promoter. The GusB promoter can provide ubiquitous gene expression, but it has been reported to give relatively weak expression. Use of the CAG and CBA promoters in the vectors of the present invention will lead to higher GBA1 expression than observed with GusB.

### Advantages of the Vectors of the Present Invention Over ERT and SRT

AAV2/9 GBA1 gene therapy with the CAG or CBA promoter should treat neurological manifestations seen with Type II and Type III patients, whereas ERT does not cross the blood-brain barrier, so currently there is no treatment for neurodegenerative forms of Gaucher disease.

AAV2/9 GBA1 gene therapy with the CAG or CBA promoter should also provide improvements over SRT. The first molecule used for SRT in Gaucher disease was the imminosugar miglustat (Zavesca®). The drug is administered orally three times a day. SRT is effective in reducing the organomegaly and the chitotriosidase activity, and ameliorating the skeletal diseases. However, it does not have major effects on the hematopathology and its administration is accompanied by persistent gastrointestinal adverse effects. Although miglustat crosses the bloodbrain barrier, no beneficial effects on the neuropathology have been reported when administered to type III patients. An alternative molecule used in SRT is eliglustat (Genzyme). The treatment demonstrates reversal of organomegaly, amelioration of anaemia and thrombocytopenia, and improvement in bone disease. However, since it does not cross the blood-brain barrier, eliglustat is not suitable for type II and type III patients.

AAV2/9 GBA1 gene therapy with the CAG or CBA promoters should treat the lung and bone since gene therapy can be considered a cure rather than disease management. ERT and SRT can be considered as methods of disease management. Although ERT is an effective treatment for the visceral aspects of Gaucher disease for type I and III patients, there is very little effect on the lungs in many patients despite being on ERT for many years. Patients experience bone pain, repeat factures and in addition, existing bone damage occurred prior to starting ERT is irreversible.

AAV2/9 GBA1 gene therapy will be given as a one-off treatment which should last for years, compared to ERT or SRT which is a lifelong treatment requiring homecare and nursing support for many patients. Indeed, patients who have been on ERT for many years report problems with accessing veins and report anxiety about this for the future.

Thus, the vectors of the present invention offer improved therapies for Gaucher disease over ERT and SRT.

### Example 2: Comparing Survival Rates of GBA1 KO Mice Following Administration of GBA1 Gene Therapy Treatment

It was hypothesised that GBA1 gene therapy could improve survival rates in GCase deficient mice. To address this, GBA1 knockout mice (K15-Inl/Inl KO) were generated by insertion of a loxP cassette in the murine GBA1 gene. At postnatal day 0-1, purified recombinant AAV vectors encoding GBA1 (AAV9.SYN.GBA1.AAV9.CAG.GBA1 or AAV9.CBA.GBA) were administered to the KO mice. Two doses of viral vectors were given, namely 2.4×10¹⁵ vg/kg or 3.3×10¹⁴ vg/kg and survival rates were monitored over 8-weeks. Wild-type and untreated GBA1 KO mice were used as controls.

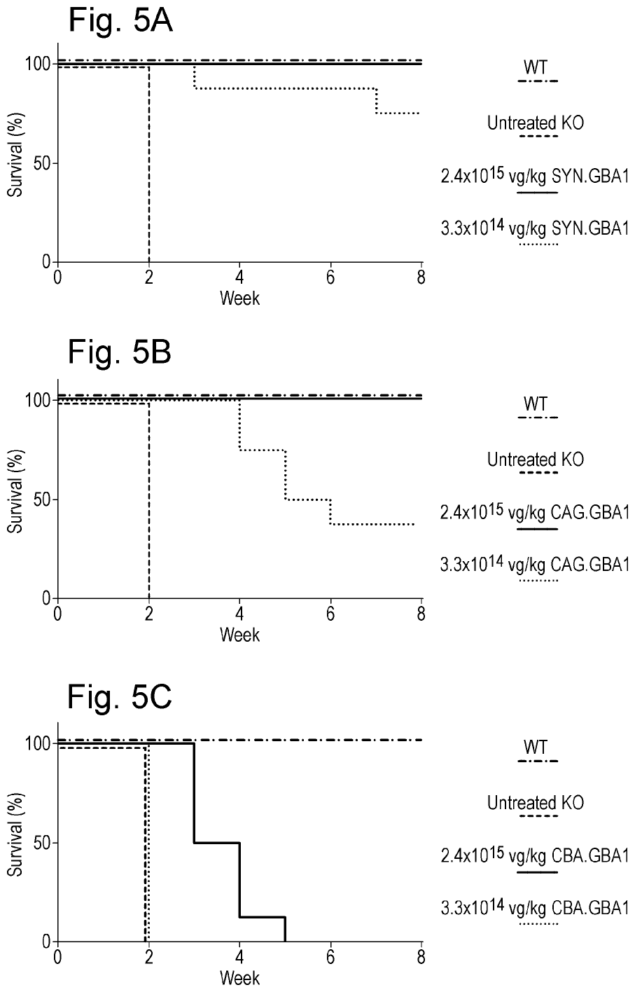

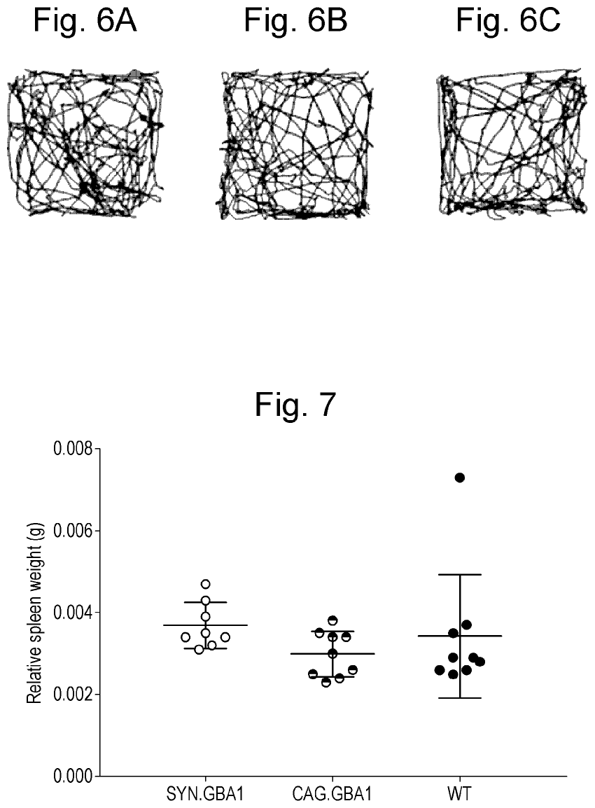

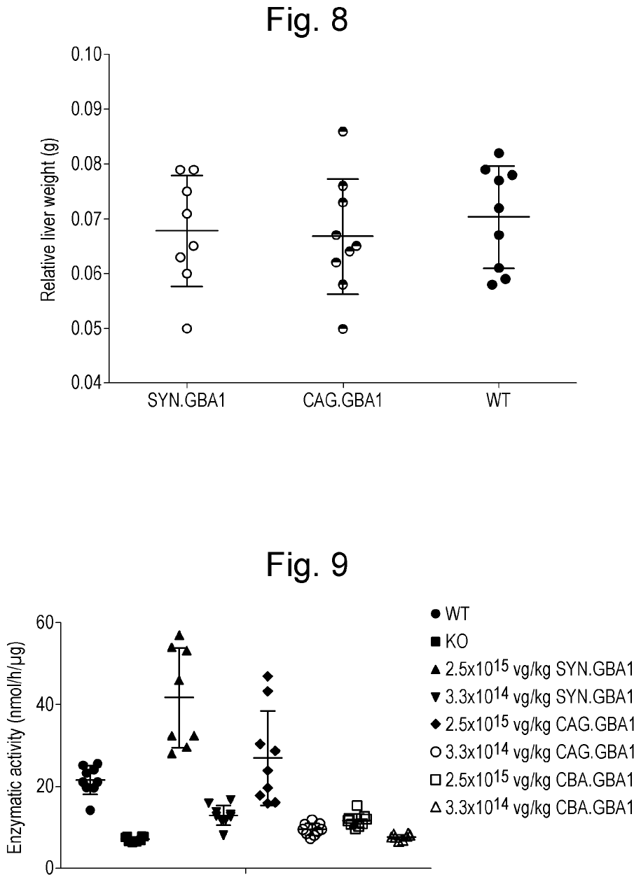

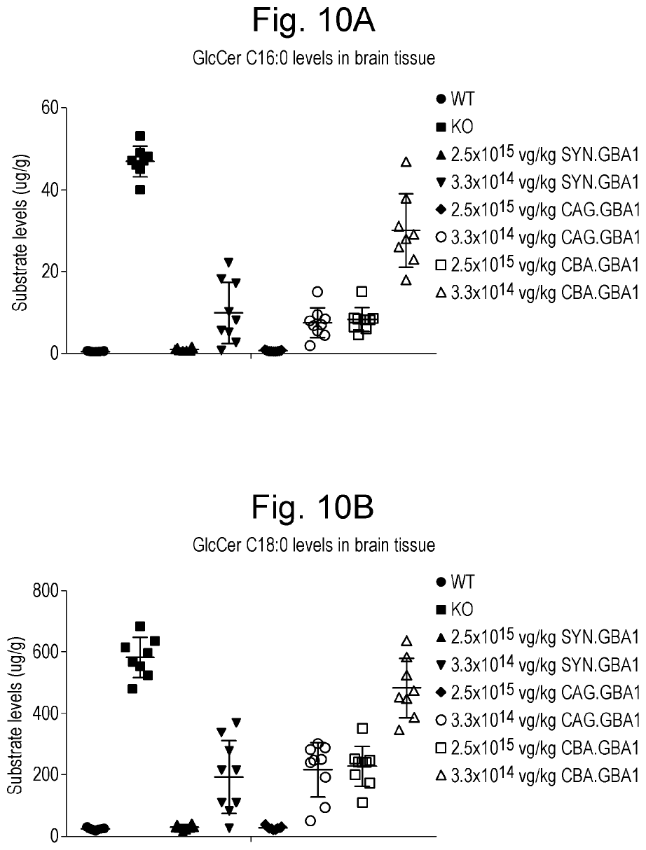

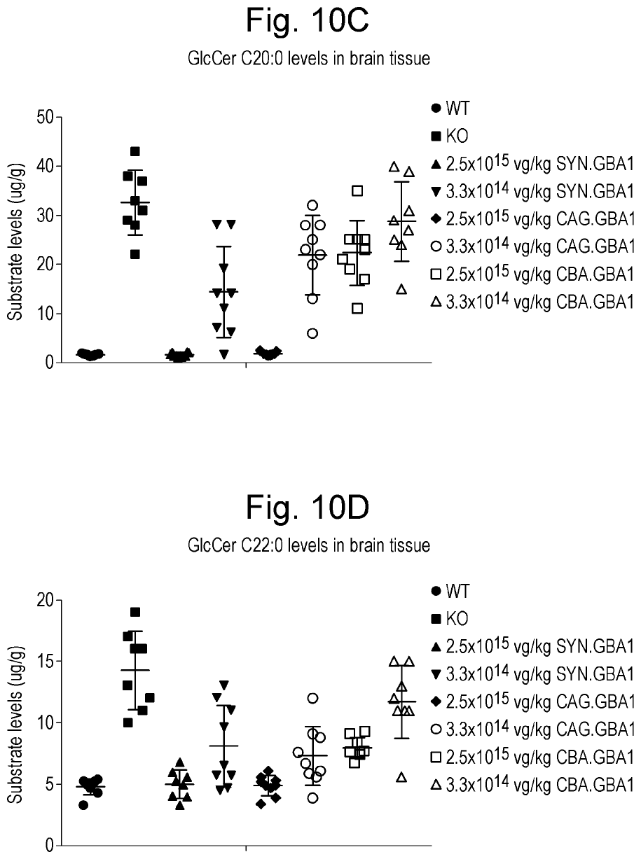

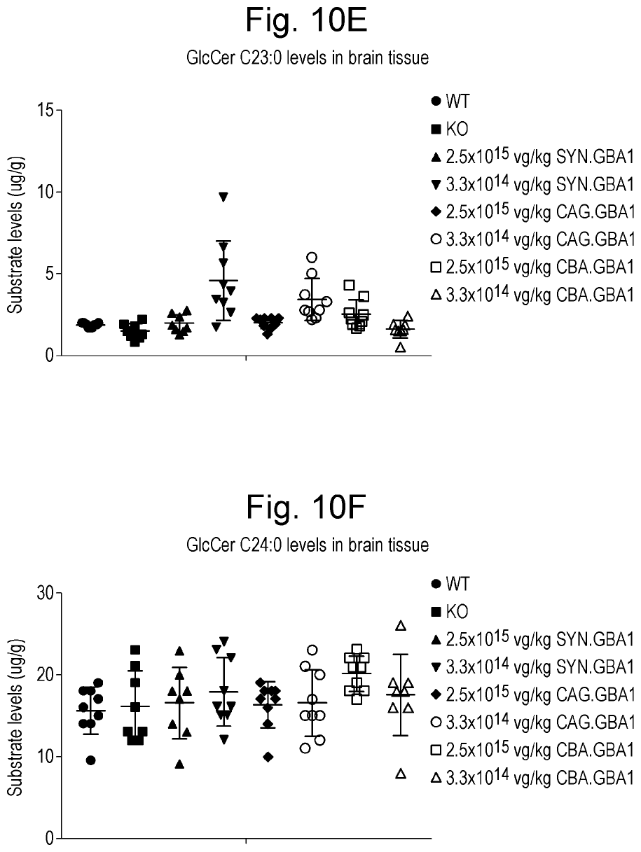

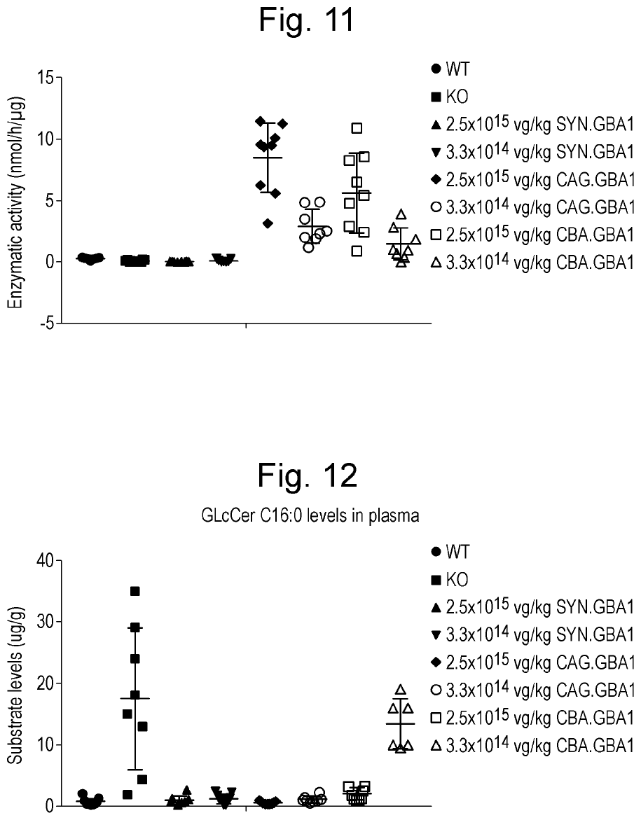

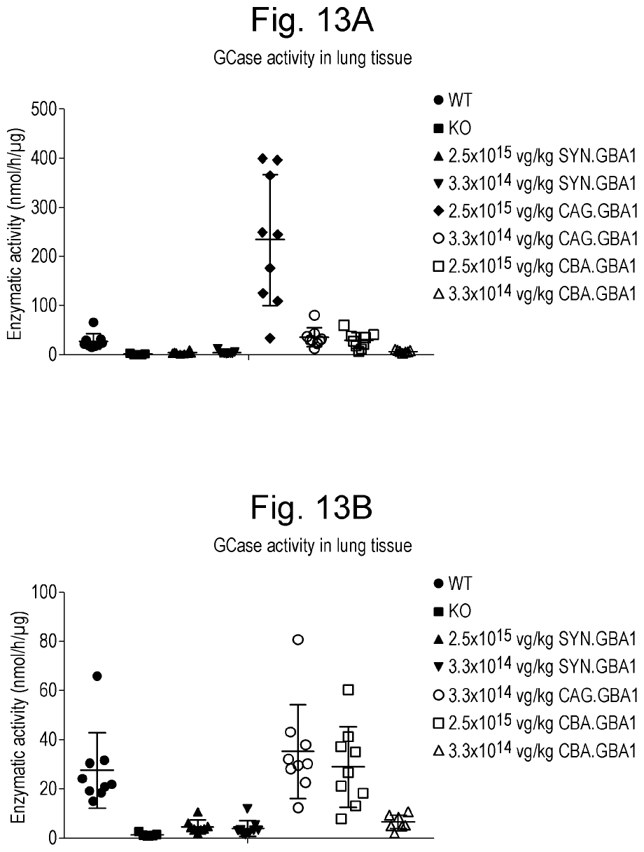

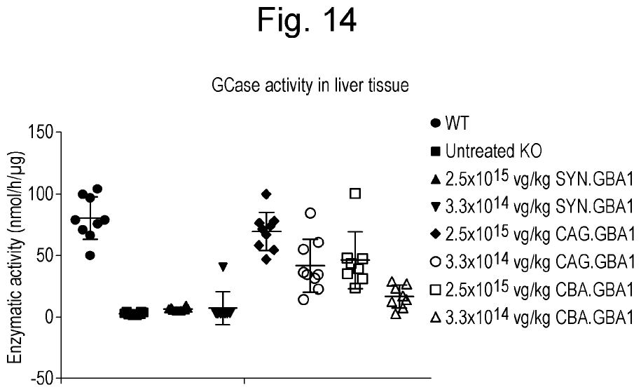

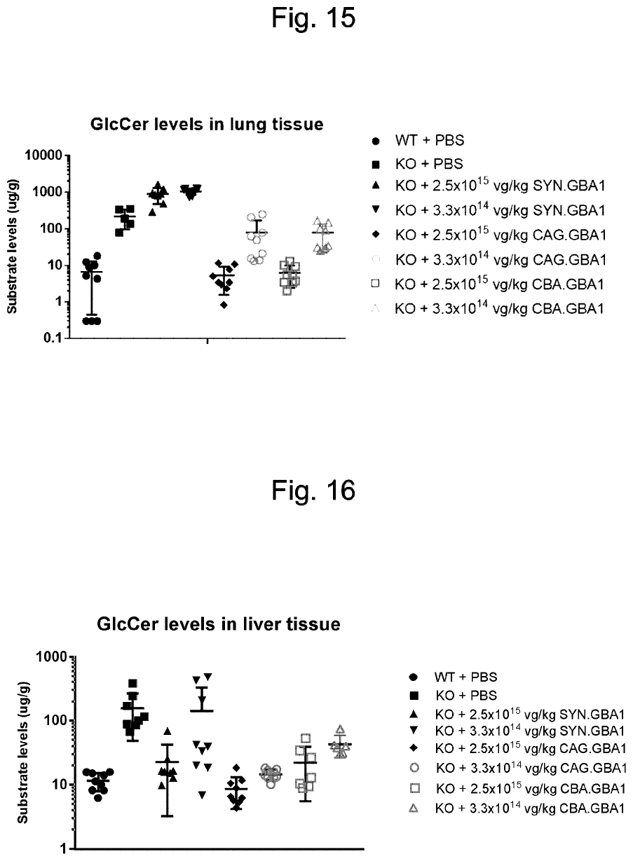

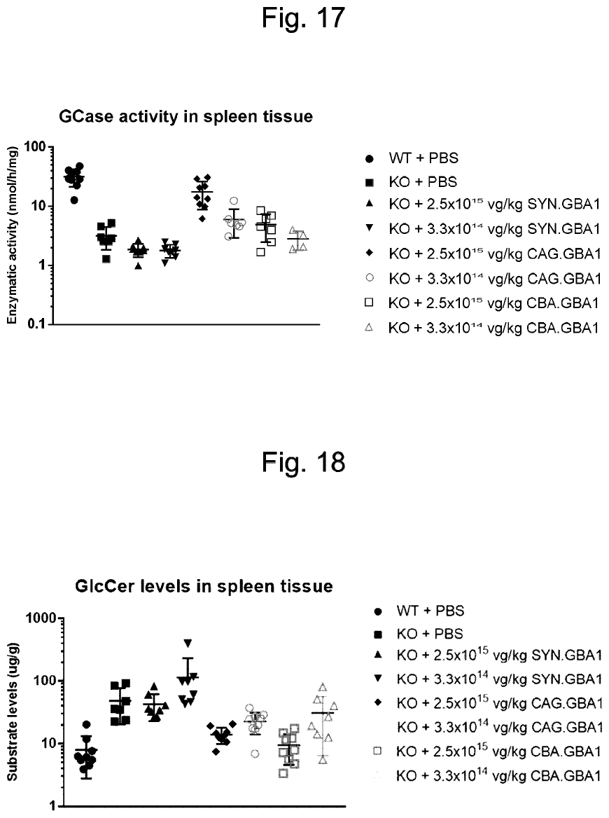

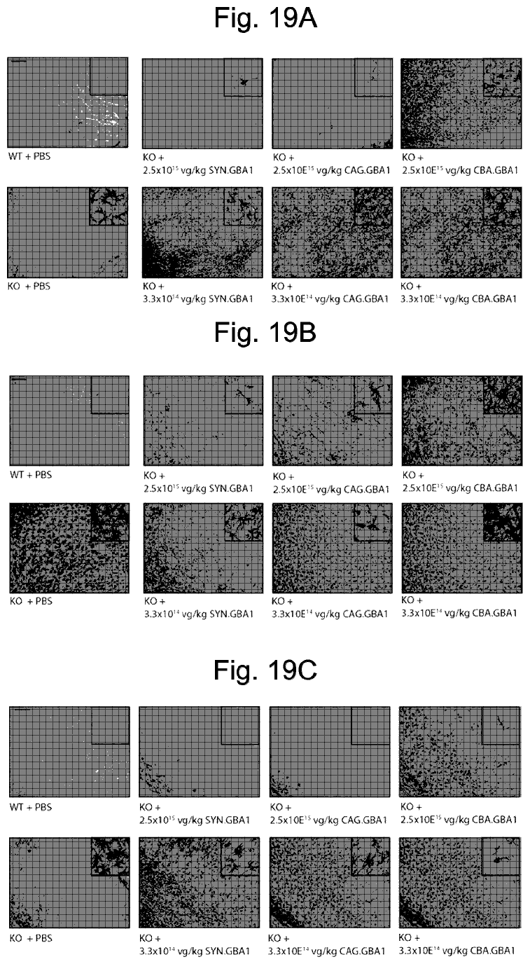

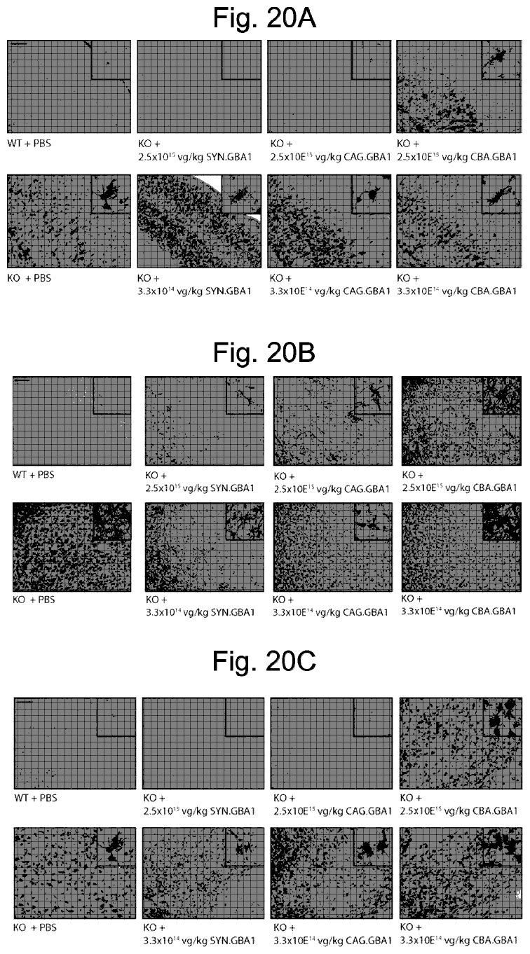

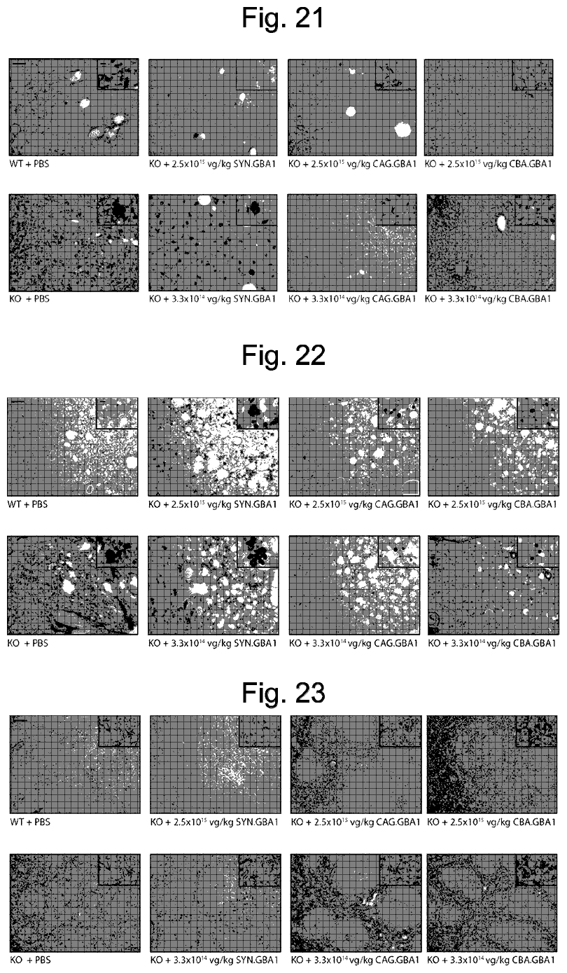

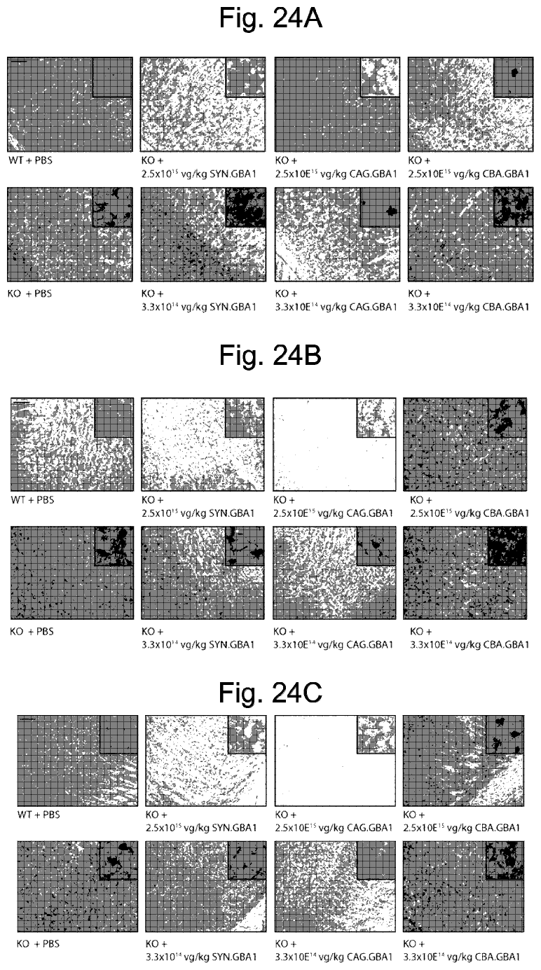

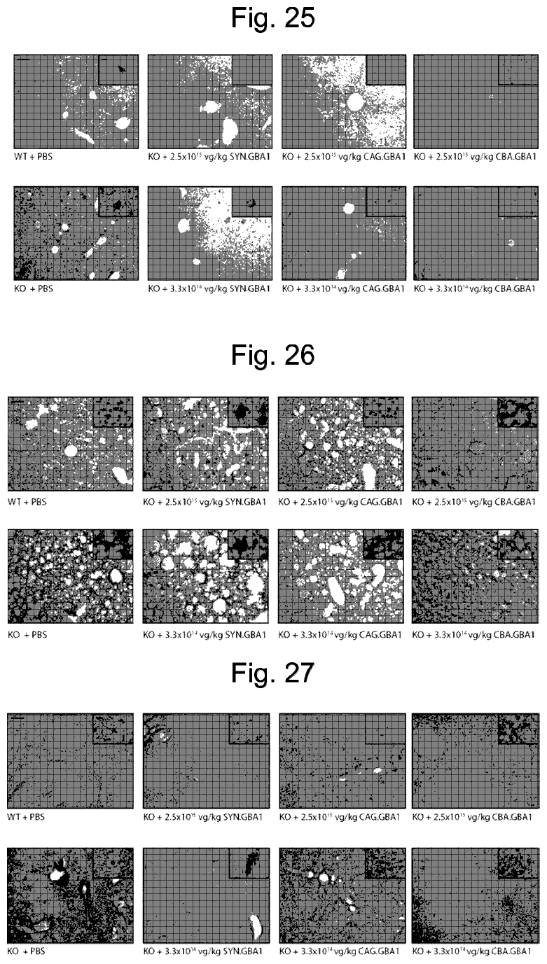

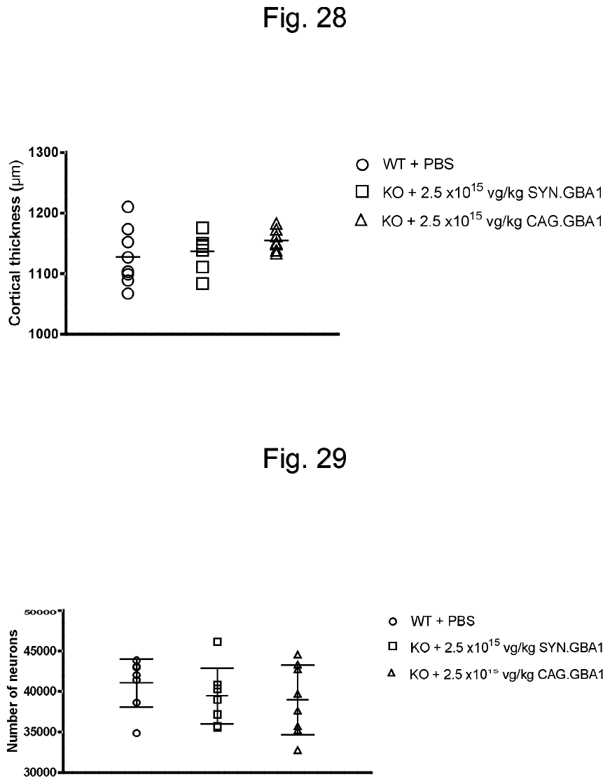

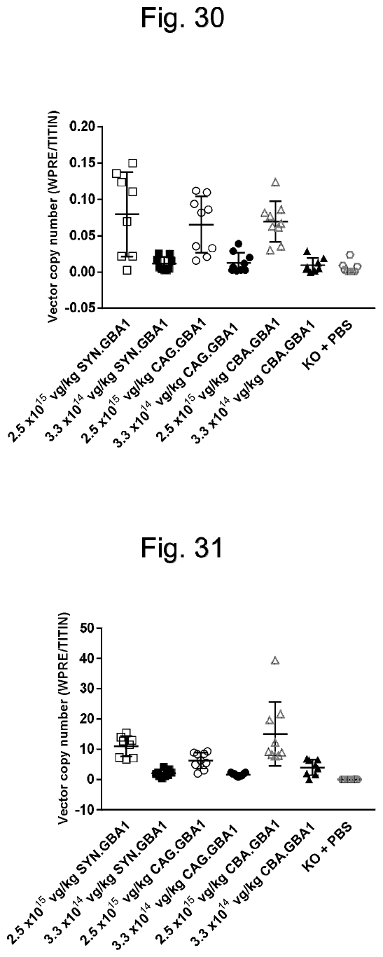

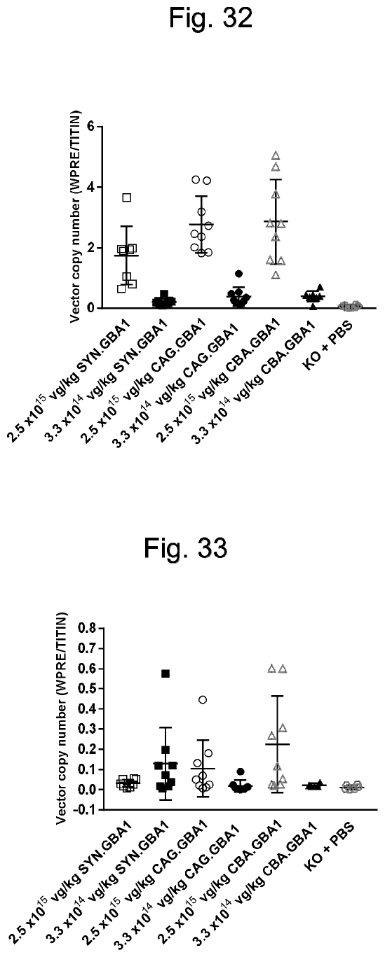

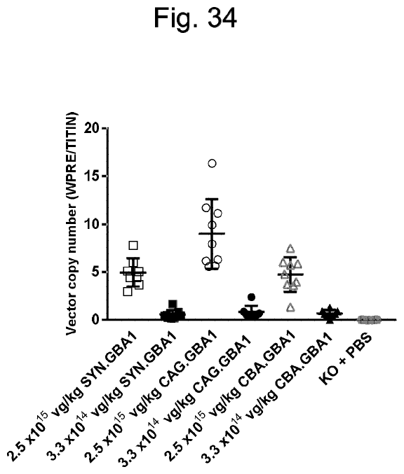

All GBA1 knockout mice exhibited improved survival rates following administration of any higher dose (2.4×10¹⁵ vg/kg) GBA1 gene therapy treatment when compared to untreated GBA1 KO mice (FIGS. 5A, 5B and 5C). Animals treated with higher dose AAV vectors encoding GBA1 under the control of the neuronal-selective (SYN) or ubiquitous (CAG) promoters exhibited the same survival rates as WT animals (FIGS. 5A and 5B). Lower dose groups treated with AAV vectors encoding GBA1 under the control of the SYN or CAG promoters had improved survival compared to KO mice but overall reduced survival compared to WT mice.

Similar observations were made in GBA1 KO mice following administration of an AAV vector encoding GBA1 under the control of a CBA promoter, with higher dose treatment of gene therapy exhibiting improved survival compared to KO mice (FIG. 5C). However, survival rates were not restored to the rates observed with WT mice. In addition, low dose administration of AAV9.CBA.GBA did not improve overall survival compared to KO mice. Thus, delivery of GBA1 gene therapy with ubiquitous (CAG or CBA) or neuronal-selective (SYN) promoters increases survival rates in mice in a dose-dependent manner. Surprisingly, despite both CAG and CBA promoters being ubiquitous, GBA1 gene therapy treatment using the CAG promoter was not only as effective as with the synapsin promoter but also provided superior survival rates compared to GBA1 gene therapy treatment with the CBA promoter, with all high dose CAG treated mice surviving to 8-weeks.

### Example 3: GBA1 Gene Therapy Treatment Normalises Motor Function and Gaucher-Associated Peripheral Physiology

To assess whether the GBA1 KO mice that received GBA1 gene therapy (AAV9.SYN.GBA1 or AAV9.CAG.GBA1) in Example 2 exhibit long-term abnormal motor function and behaviour, which is typical of GCase deficient animals, the open field activity of the mice was traced at postnatal day 56 (FIG. 6). To track mouse movement, each mouse was placed in a square transparent Plexiglass chamber for 5 minutes for free exploration prior to 5 minutes filmed movement. All traces were comparable, confirming that mice given IV administration of AAV9.SYN.GBA1 or AAV9.CAG.GBA1 gene therapy maintain normal motor coordination and behaviour.

Liver and spleen enlargement is also a common manifestation of Gaucher disease due to the accumulation of Gaucher cells in these organs. Measurements of mouse spleen and liver weights confirmed that GBA1 KO mice that received GBA1 gene therapy (AAV9.SYN.GBA1 or AAV9.CAG.GBA1) maintain normal Gaucher-associated peripheral physiology (FIGS. 7 and 8). All spleen and liver weights were adjusted for body weight and revealed no significant difference in weights between GBA gene therapy treated (AAV9.SYN.GBA1 or AAV9.CAG.GBA1) KO mice and WT mice.

Taken together, these results indicate that GBA1 gene therapy treatment normalises Gaucher associated peripheral physiology, motor coordination and behaviour in mice.

### Example 4: Measuring GCase Enzyme Activity and Glucosylceramide Substrate Levels in Brain Homogenate

To establish whether GBA1 gene therapy directly restores GCase enzyme activity, a GCase enzyme assay was performed as described above in brain tissue. Briefly, homogenised tissue samples were incubated with a synthetic substrate (4-methylumbelliferone-β-glucopyranoside) for 2 hours at 37° C. prior to fluorescence detection. This confirmed that higher dose GBA1 gene therapy using AAV9.SYN.GBA1 or AAV9.CAG.GBA1 increases GCase enzyme activity to at least WT levels in brain tissue (FIG. 9).

Consistent with a restoration of GCase enzyme activity, measurements of glucosylceramide substrate levels by mass spectrometry analysis confirmed that GCase substrate levels were restored to basal levels following GBA1 gene therapy in GBA1 KO mice (FIGS. 10A, 10B, 10C, 10D and 10E). Specifically, GlcCer C16:0, GlcCer C18:0, GlcCer C20:0 and GlcCer C22:0 analyte levels, which were elevated in GBA1 KO mice, were restored to WT levels following GBA1 gene therapy using AAV9.SYN.GBA1 or AAV9.CAG.GBA1. Mice treated with the gene therapy vector encoding GBA1 under the control of the CBA promoter (AAV9.CBA.GBA) demonstrated an overall reduction in GlcCer C16:0, GlcCer C18:0, GlcCer C20:0 and GlcCer C22:0 substrate levels, albeit with less dramatic reductions in substrate levels than were observed in mice administered AAV9.SYN.GBA1 or AAV9.CAG.GBA1. GlcCer C23:0 and GlcCer C24:0 analyte levels remained largely unaffected in GBA1 KO mice, thus there were few changes between the gene therapy treated mice and untreated control mice (FIG. 10F).

These results confirm that the gene therapy vectors of the present invention are expressed in neuronal cells where they reduce the accumulation of GCase substrates in GBA1 KO mice, suggesting that the vectors of the present invention are able to cross the blood-brain barrier and could be useful in the treatment of neurological manifestations seen with Type 2 and Type 3 Gaucher disease patients.

### Example 5: Measuring GCase Enzyme Activity and Glucosylceramide Substrate Levels in Plasma

Having confirmed that the vectors of the present invention can restore GCase activity in brain tissue, it was important to assess whether the gene therapy vectors can restore peripheral GCase activity. As above, plasma samples isolated from WT, GBA1 KO and GBA1 gene therapy treated mice were incubated with the synthetic substrate prior to GCase activity determination by fluorescence detection. This identified an increase in systemic GCase enzyme activity in plasma following AAV9.CAG.GBA or AAV9.CBA.GBA vector gene therapy treatment (FIG. 11). In contrast, there was no change in the systemic activity of the GCase enzyme in GBA1 KO mice following administration of an AAV vector encoding GBA1 under the control of the SYN promoter. Although, all three promoters restored the GCase substrate GlcCer C16:0 to basal levels (FIG. 12).

Despite a minimal change in GCase enzyme activity in plasma from GBA1 KO mice and low dose AAV9.CBA.GBA treated mice, there was an accumulation of GlcCer C16:0 substrate levels in these animals (FIG. 12). All other treatment groups demonstrated GCase substrate levels equivalent to WT mice.

Thus, the expression constructs of the present invention promote increased GCase enzyme activity in plasma, which was not observed when vectors encoding GBA1 under the control of the SYN promoter were administered.

### Example 6: Measuring GCase Enzyme Activity Levels in Lung Homogenate and Liver Homogenate

GCase enzyme activity was assessed in lung tissue as previously described. This demonstrated that higher dose GBA1 gene therapy using the AAV9.CAG.GBA vector confers supramaximal GCase enzyme activity levels compared to other treatment groups (FIG. 13A). Furthermore, lower dose GBA1 gene therapy using the AAV9.CAG.GBA vector and higher dose GBA1 gene therapy using the AAV9.CBA.GBA vector also increased GCase enzyme activity to WT levels (FIG. 13B). In contrast, mice administered the AAV9.SYN.GBA vector did not restore GCase enzyme activity.

The liver is a major organ for GCase enzyme production and Gaucher disease. Thus, GCase enzyme activity was measured in liver tissue following GBA1 gene therapy treatment (FIG. 14). Loss of GBA1 in KO mice resulted in an observable decrease in GCase activity. This activity was not restored following administration of an AAV vector encoding GBA1 under the control of the SYN promoter. In contrast, administration of higher dose AAV9.CAG.GBA or AAV9.CBA.GBA increased GCase enzyme levels, with complete restoration of GCase enzyme activity to WT levels upon treatment with GBA1 under the control of the CAG promoter.

These results indicate that administration of GBA1 under the control of the SYN promoter fails to alleviate deficiencies in GCase enzyme activity observed in GBA1 KO mice in lung or liver tissues. In contrast, GBA1 gene therapy treatment using expression constructs under the control of the CAG or CBA promoters can increase, if not completely restore GCase enzyme activity in these organs.

### Example 7: Measuring Glucosylceramide Substrate Levels in Lung Homogenate and Liver Homogenate

Having verified that the vectors of the present invention can restore GCase activity in lung and liver tissues, total GlcCer substrate levels (including GlcCer C16:0, GlcCer C18:0. GlcCer C20:0, GlcCer C22:0, GlcCer C23:0 and GlcCer C24:0) were measured in lung and liver homogenates following GBA1 gene therapy. In lung tissue, higher dose administration of GBA1 using the expression constructs of the present invention restored total GlcCer substrate levels to wildtype levels (FIG. 15), which was consistent with the restoration of GCase activity observed in lung tissue following GBA1 gene therapy using the AAV9.CAG.GBA or AAV9.CBA.GBA vectors (FIG. 13A and FIG. 13B). A reduction in total GlcCer substrate levels was also observed following lower dose administration of GBA1 using the expression constructs of the present invention. In contrast, GBA1 gene therapy using expression constructs under the control of the SYN promoter exaggerated the accumulation of GlcCer substrate levels in the lung compared to GBA1 KO mice (FIG. 15).

Similar observations were made in liver tissue whereby GlcCer substrate levels decreased in response to GBA1 gene therapy using the vectors of the present invention (FIG. 16). Both higher and lower doses of AAV9.CAG.GBA restored GlcCer substrate levels to wildtype levels, while administration of higher and lower doses of AAV9.CBA.GBA to GBA1 KO mice reduced total GlcCer substrate accumulation. Despite the minimal change in GCase activity following AAV9.SYN.GBA gene therapy in liver tissue, administering a high dose of this expression vector under the control of the SYN promoter resulted in a minor reduction in total GlcCer levels.

These results demonstrate that expression constructs of the present invention successfully reduce lung and liver GlcCer substrate accumulation in GBA1 KO mice at both higher (2.5×10¹⁵ vg/kg) and lower (3.3×10¹⁴ vg/kg) doses, whereas administration of GBA1 under the control of the SYN promoter fails to restore GlcCer substrate levels to wildtype levels. In fact. GBA1 gene therapy using AAV9.SYN.GBA negatively exaggerated GlcCer substrate accumulation in the lung.

### Example 8: Measuring GCase Enzyme Activity and Glucosylceramide Substrate Levels in Spleen Tissue

Gaucher cell accumulation is frequently observed in the spleen during Gaucher disease. Thus, GCase enzyme activity was assessed in spleen tissue as previously described (FIG. 17). As above, spleen samples isolated from WT, GBA1 KO and GBA1 gene therapy treated mice were incubated with the synthetic substrate prior to GCase activity determination by fluorescence detection. This identified an increase in GCase enzyme activity in the spleen following AAV9.CAG.GBA or AAV9.CBA.GBA vector gene therapy treatment compared to GBA1 KO mice (FIG. 17). Consistent with a restoration of GCase enzyme activity, measurements of glucosylceramide substrate levels by mass spectrometry analysis confirmed that total GCase substrate levels (including GlcCer C16:0. GlcCer C18:0, GlcCer C20:0, GlcCer C22:0. GlcCer C23:0 and GlcCer C24:0) were reduced following GBA1 gene therapy in GBA1 KO mice (FIG. 18).

In contrast, there was no change in the systemic activity of the GCase enzyme in GBA1 KO mice following administration of an AAV vector encoding GBA1 under the control of the SYN promoter. Rather, the loss of GCase enzyme activity in GBA1 KO mice, coupled with the accumulation of total GlcCer substrate, was exaggerated by AAV9.SYN.GBA gene therapy (FIG. 17 and FIG. 18).

Together these results confirm that GBA1 gene therapy using the expression vectors of the present invention will be suitable for the treatment of all forms of Gaucher disease due to their activity in a wide range of tissues, including brain, plasma, lung, spleen and liver.

### Example 9: Examining Astrocyte Activation in Brain Tissue

Patients with neuronopathic Gaucher disease display astrogliosis. Accordingly, an increase in the expression of the astrocyte marker, GFAP, was observed in GBA1 KO mice, mimicking the neuroinflammation seen during Gaucher disease. Consistent with a restoration of GCase enzyme activity and GlcCer substrate levels in brain tissue in FIG. 9, higher dose GBA1 gene therapy using AAV9.SYN.GBA or AAV9.CAG.GBA restored GFAP expression levels to levels comparable to WT mice in the Ventral post medial/ventral post lateral thalamic nuclei (VPM/VPL) region (FIG. 19A), the Gigantocellular nuclei (Gi) region (FIG. 19B) and the Somato-barrel field 1 (SIBF) region (FIG. 19C). Thus, GBA1 gene therapy prevents the neuronopathic consequences of GBA1 loss.

### Example 10: Examining Macrophage Activation and Lysosomal Pathology in Brain, Liver, Lung and Spleen Tissue

Widespread accumulation of activated, glucosylceramide laden macrophages is a pathophysiological consequence of Gaucher disease. Macrophage activation was assessed by examining the abundance of the macrophage marker, CD68, in brain (FIG. 20A, B C), liver (FIG. 21), lung (FIG. 22) and spleen (FIG. 23) tissues. In brain tissue, CD68 expression levels were reduced in GBA1 KO mice treated with high dose AAV9.SYN.GBA or AAV9.CAG.GBA compared to untreated GBA1 KO mice. In the liver, gene therapy using expression constructs under the control of the CAG, CBA or SYN promoters caused a reduction in the levels of CD68 expression, which was comparable to WT liver sections following high dose AAV9.SYN.GBA or AAV9.CAG.GBA (FIG. 21). In the lung, gene therapy with all three vectors reduced CD68 expression. Importantly, treatment with AAV9.CAG.GBA caused a significant reduction in CD68 expression and overall macrophage morphology, which was consistent with the supramaximal GCase activity measured in FIG. 13A (FIG. 22). Furthermore, high dose administration of AAV9.CBA.GBA1 resulted in the enhanced reduction of CD68 staining compared to AAV9.SYB.GBA1 treatment in the lung, with CD68 positive cells appearing smaller in morphology. In the spleen, untreated KO mice exhibited abnormal accumulation of macrophages in the white pulp containing enlarged CD68 positive cells. This enlargement was not visible in high dose AAV9.CAG.GBA treated GBA1 KO mice (FIG. 23).

Other pathological consequences of Gaucher disease include the accumulation of GlcCer substrates in lysosomes, resulting in a swollen, enlarged morphology. These enlarged lysosomes were visible by Lamp-1 staining in the brain tissue (FIG. 24A, B, C), liver tissue (FIG. 25), lung tissue (FIG. 26) and spleen tissue (FIG. 27) of GBA1 KO mice due to the accumulation of GlcCer substrate levels measured in these tissues. In the brain, IV administration of higher dose (2.5×10¹⁵ vg/kg) AAV9.SYN.GBA1 and AAV9.CAG.GBA1 resulted in reduced cellular lysosomal content comparable to wild-type levels in the Somato-barrel field 1 region (FIG. 24A), the Gigantocellular nuclei region (FIG. 24B) and the ventral post medial/ventral post lateral thalamic nuclei region (FIG. 24C). Gene therapy treatment with high and low doses of AAV9.CAG.GBA1 prevented the accumulation of enlarged lysosomes in the liver (FIG. 25) the lung (FIG. 26) and the spleen (FIG. 27). A partial reduction in the number of enlarged lysosomes, as assessed by Lamp-1 staining, was visible in liver (FIG. 25) and spleen (FIG. 27) tissue following administration of any of AAV9.CAG.GBA1, AAV9.CBA.GBA1 or AAV9.SYN.GBA1.

### Example 11: Neuronal Counts and Cortical Thickness Measurements

Changes in cortical thickness and severe neuronal loss are frequently observed during neuronopathic forms of Gaucher disease. Thus, cortical thickness was assessed in GBA1 KO mice treated with AAV9.SYN.GBA or AAV9.CAG.GBA (FIG. 28). This confirmed that gene therapy in GBA1 KO mice using expression constructs under the control of the SYN or CAG promoters maintained cortical region thickness at comparable levels to WT animals. Furthermore, these mice did not exhibit neuronal loss, as assessed using neuronal counts by stereology (FIG. 29).

### Example 12: Investigating Viral Copy Number in Brain, Liver, Lung, Spleen and Heart Tissues in Gba1 KO Mice Treated with GBA1 Gene Therapy

Viral copy number was evaluated in brain (FIG. 30), liver (FIG. 31), lung (FIG. 32), spleen (FIG. 33) and heart (FIG. 34) tissue in Gba1 KO mice treated with GBA1 gene therapy by quantitative PCR. This confirmed that higher dose GBA1 gene therapy using any of the AAV9.SYN.GBA, AAV9.CAG.GBA or AAV9.CBA.GBA vectors results in increased vector copy number in the brain, liver, lung, spleen on heart compared to lower dose GBA1 gene therapy.

Overall, these results demonstrate that GBA1 gene therapy expression vectors of the present invention are active in a wide range of tissues and reduce pathophysiology associated with Gaucher disease, hence will be suitable to treat patients suffering from all forms of Gaucher disease.

### REFERENCES

Altschul S. F. A protein alignment scoring system sensitive at all evolutionary distances J. Mol. Evol. 36, 290-300 (1993).
Altschul, S. F. et al., Basic local alignment search tool J. Mol. Biol., 215, 403-410 (1990).
Ayuso E. et al. High AAV vector purity results in serotype- and tissue-independent enhancement of transduction efficiency. Gene Ther. 17, 503-510 (2010).
Coura Rdos S. and Nardi, N. B., The state of the art of adeno-associated virus-based vectors in gene therapy, Virol. J., 4, 99 (2007).
Devereux, J. et al. A comprehensive set of sequence analysis programmes for the VAX. Nucl. Acids Res. 12, 387-395 (1984).
Dong, J. Y. et al., Quantitative analysis of the packaging capacity of recombinant adeno-associated virus, Hum. Gene Ther., 7, 2101-2112 (1996).
Erles, K., Sebokova, P. and Schlehofer, J. R., Update on the prevalence of serum antibodies (IgG and IgM) to adeno-associated virus (AAV), J Med. Virol. 59, 406-411 (1999).
Henikoff, S. and Henikoff, J. G., Proc. Natl. Acad. Sci. USA 89, 10915-10919 (1992).
Hermonat, P. L. et al., Genetics of adeno-associated virus: isolation and preliminary characterization of adeno-associated virus type 2 mutants, J. Virol., 51, 329-339 (1984).
Karlin, S. and Altschul, S.F., Applications and statistics for multiple high-scoring segments in molecular sequences Proc. Natl. Acad. Sci. USA 90: 5873-5787 (1993).
Kotin, R. M. et al., Site-specific integration by adeno-associated virus, Proc. Natl. Acad. Sci U.S.A 87, 2211-2215 (1990).
Manno, C. S. et al., Successful transduction of liver in hemophilia by AAV-Factor IX and limitations imposed by the host immune response, Nat. Med. (2006).
Michelfelder, S. and Trepel, M., Adeno-associated viral vectors and their redirection to cell-type specific receptors, Adv. Genet., 67, 29-60 (2009).
Moskalenko, M. et al., Epitope mapping of human anti-adeno-associated virus type 2 neutralizing antibodies: implications for gene therapy and virus structure, J. Virol., 74, 1761-1766 (2000).
Summerford, C. and Samulski, R. J., Membrane-associated heparan sulfate proteoglycan is a receptor for adeno-associated virus type 2 virions, J. Virol., 72, 1438-1445 (1998).
Sun, J. Y. et al., Immune responses to adeno-associated virus and its recombinant vectors, 10, 964-976 (2003).
Thomas, C. E. et al., Rapid uncoating of vector genomes is the key to efficient liver transduction with pseudotyped adeno-associated virus vectors, J. Virol. 78, 3110-3122 (2004).
Vincent-Lacaze, N. et al., Structure of adeno-associated virus vector DNA following transduction of the skeletal muscle, J Virol. 73, 1949-1955 (1999).
Wenger D. A. et al. Synthetic substrate beta-glucosidase activity in leukocytes: a reproducible method for the identification of patients and carriers of Gaucher's disease. Clin Genet. 13, 145-153 (1978).
Wu, Z. et al., Adeno-associated virus serotypes: vector toolkit for human gene therapy, Mol. Ther., 14, 316-327 (2006).
Zaiss, A. K. and Muruve, D. A., Immunity to adeno-associated virus vectors in animals and humans: a continued challenge. Gene Ther 15, 808-816 (2008).

1. An expression construct comprising in a 5′ to 3′ direction:
(a) the CBA promoter as shown in SEQ ID NO: 2, or a sequence having at least 90% sequence identity to SEQ ID NO: 2 that retains the ability to express GBA1; and
(b) (i) the GBA1 sequence as shown in SEQ ID NO: 1, or a sequence having at least 70% sequence identity to SEQ ID NO: 1 that retains the functionality of GBA1; or (ii) a GBA1 sequence encoding the polypeptide as shown in SEQ ID NO: 12 or a polypeptide having at least 90% sequence identity to the polypeptide of SEQ ID NO: 12 that retains the functionality of GBA1.

2. An expression construct comprising in a 5′ to 3′ direction:
(a) the CAG promoter as shown in SEQ ID NO: 3, or a sequence having at least 90% sequence identity to SEQ ID NO: 3 that retains the ability to express GBA1; and
(b) (i) the GBA1 sequence as shown in SEQ ID NO: 1, or a sequence having at least 70% sequence identity to SEQ ID NO: 1 that retains the functionality of GBA1; or (ii) a GBA1 sequence encoding the polypeptide as shown in SEQ ID NO: 12 or a polypeptide having at least 90% sequence identity to the polypeptide of SEQ ID NO: 12 that retains the functionality of GBA1.

3. The expression construct of claim 1, wherein the construct comprises SEQ ID NO: 5, or a sequence having at least 90% sequence identity to SEQ ID NO: 5 that retains the ability to express a functional form of GBA1.

4. The expression construct of claim 2, wherein the construct comprises SEQ ID NO: 6 or SEQ ID NO: 20, or a sequence having at least 90% sequence identity to SEQ ID NO: 6 or SEQ ID NO: 20 that retains the ability to express a functional form of GBA1.

5. The expression construct of claim 1, comprising in a 5′ to 3′ direction:
(a) the CBA promoter as shown in SEQ ID NO: 2, or a sequence having at least 90% sequence identity to SEQ ID NO: 2 that retains the ability to express GBA1;
(b) (i) the GBA1 sequence as shown in SEQ ID NO: 1, or a sequence having at least 70% sequence identity to SEQ ID NO: 1 that retains the functionality of GBA1; or (ii) a GBA1 sequence encoding the polypeptide as shown in SEQ ID NO: 12 or a polypeptide having at least 90% sequence identity to the polypeptide of SEQ ID NO: 12 that retains the functionality of GBA1; and
(c) the WPRE sequence as shown in SEQ ID NO: 4, or a sequence having at least 90% sequence identity to SEQ ID NO: 4 that retains the functionality of the WPRE sequence.

6. The expression construct of claim 2, comprising in a 5′ to 3′ direction:
(a) the CAG promoter as shown in SEQ ID NO: 3, or a sequence having at least 90% sequence identity to SEQ ID NO: 3 that retains the ability to express GBA1;
(b) (i) the GBA1 sequence as shown in SEQ ID NO: 1, or a sequence having at least 70% sequence identity to SEQ ID NO: 1 that retains the functionality of GBA1; or (ii) a GBA1 sequence encoding the polypeptide as shown in SEQ ID NO: 12 or a polypeptide having at least 90% sequence identity to the polypeptide of SEQ ID NO: 12 that retains the functionality of GBA1; and
(c) the WPRE sequence as shown in SEQ ID NO: 4, or a sequence having at least 90% sequence identity to SEQ ID NO: 4 that retains the functionality of the WPRE sequence.

7. The expression construct of claim 5, wherein the construct comprises SEQ ID NO: 7, or a sequence having at least 90% sequence identity to SEQ ID NO: 7 that retains the ability to express a functional form of GBA1.

8. The expression construct of claim 6, wherein the construct comprises SEQ ID NO: 8 or SEQ ID NO: 21, or a sequence having at least 90% sequence identity to SEQ ID NO: 8 or SEQ ID NO: 21 that retains the ability to express a functional form of GBA1.

9. The expression construct of any one of claims 1, 2, 5 or 6, wherein the sequence of (b)(i) has at least 80% sequence identity to SEQ ID NO: 1.

10. The expression construct of claim 9, wherein the sequence of (b)(i) has at least 90% sequence identity to SEQ ID NO: 1.

11. The expression construct of any one of claims 1, 2, 5 or 6, wherein the sequence of (b)(ii) has at least 95% sequence identity to SEQ ID NO: 12.

12. The expression construct of any one of claims 1, 2, 5 or 6, wherein the GBA1 sequence encoding the polypeptide of SEQ ID NO: 12 is SEQ ID NO: 13, SEQ ID NO: 14, SEQ ID NO: 15 or SEQ ID NO: 16.

13. A vector comprising the expression construct according to any one of claims 1 to 12.

14. The vector according to claim 13, which is a viral vector.

15. The vector according to claim 14, which is an adeno-associated virus (AAV) vector or comprises an AAV genome or a derivative thereof.

16. The vector according to claim 15, wherein said derivative is a chimeric, shuffled or capsid modified derivative.

17. The vector according to claims 15 or 16, wherein said AAV genome is from a naturally derived serotype or isolate or clade of AAV.

18. The vector according to claim 17, wherein said AAV genome is from AAV serotype 2 (AAV2), AAV serotype 4 (AAV4), AAV serotype 5 (AAV5) or AAV serotype 8 (AAV8) and/or wherein the capsid is derived from AAV9.

19. The vector according to claim 18, wherein the genome is derived from AAV2 and the capsid is derived from AAV9.

20. The vector according to claim 19, which comprises the sequence of:
(a) SEQ ID NO: 9 or SEQ ID NO: 17, or a sequence having at least 90% sequence identity to SEQ ID NO: 9 or SEQ ID NO: 17 that retains the ability to express a functional form of GBA1; or
(b) SEQ ID NO: 10 or SEQ ID NO: 18, or a sequence having at least 90% sequence identity to SEQ ID NO: 10 or SEQ ID NO: 18 that retains the ability to express a functional form of GBA1.

21. A host cell that contains a vector of claim 13 or produces a viral vector of any one of claims 14 to 20.

22. The cell according to claim 21 that is a HEK293 or HEK293T cell.

23. A pharmaceutical composition comprising a vector of any one of claims 14 to 20 and a pharmaceutically acceptable carrier.

24. The vector according to any one of claims 14 to 20 for use in a method of preventing or treating Gaucher disease.

25. Use of a vector according to any one of claims 14 to 20 in the manufacture of a medicament for the treatment or prevention of Gaucher disease.

26. A method of treating or preventing Gaucher disease in a patient in need thereof, comprising administering a therapeutically effective amount of a vector according to any one of claims 14 to 20 to said patient.

27. The vector for use according to claim 24, the use of claim 25, or the method according to claim 26, wherein the disease to be treated is Gaucher disease Type I, II or III, or neuronopathic Gaucher disease.

28. The vector for use according to claim 24 or 27, the use of claim 25 or 27, or the method according to claim 26 or 27, wherein the treatment of Gaucher disease includes the treatment of the lungs and/or bones of a patient.

29. The vector according to any one of claims 14 to 20 for use in a method of preventing or treating lysosomal storage disorders such as Niemann-Pick disease type C (NPC), or synucleinopathies such as Parkinson's disease, dementia with Lewy bodies, multiple system atrophy and pure autonomic failure (PAF).

30. Use of a vector according to any one of claims 14 to 20 in the manufacture of a medicament for the treatment or prevention of lysosomal storage disorders such as Niemann-Pick disease type C (NPC), or synucleinopathies such as Parkinson's disease, dementia with Lewy bodies, multiple system atrophy and pure autonomic failure (PAF).

31. A method of treating or preventing lysosomal storage disorders such as Niemann-Pick disease type C (NPC), or synucleinopathies such as Parkinson's disease, dementia with Lewy bodies, multiple system atrophy and pure autonomic failure (PAF) in a patient in need thereof, comprising administering a therapeutically effective amount of a vector according to any one of claims 14 to 20 to said patient.

32. The vector for use according to claim 24, 27 or 28, the use of claim 25, 27 or 28, or the method according to claim 26, 27 or 28, wherein the vector is administered parentally, preferably intravenously or intracerebroventricularly, to a patient.

33. The vector for use according to claim 29, the use of claim 30, or the method according to claim 31, wherein the vector is administered parentally, preferably intravenously or intracerebroventricularly, to a patient.

34. The vector for use according to claim 33, the use of claim 30 or 33, or the method according to claim 31 or 33, wherein the disease to be treated is Parkinson's disease.


SEQUENCE LISTING (21 sequences)

SEQ ID NO: 1 - DNA, 1611 bp, Homo sapiens
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 2 - DNA, 260 bp, Artificial Sequence
  CBA promoter
ccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcg

SEQ ID NO: 3 - DNA, 643 bp, Artificial Sequence
  CAG promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 609 bp, Artificial Sequence
  WPRE element
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 5 - DNA, 1908 bp, Artificial Sequence
  CBA GBA1 expression construct
ccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggtaccggatcctctagagtcgactccggagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 6 - DNA, 2291 bp, Artificial Sequence
  CAG GBA1 expression construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggtaccggatcctctagagtcgactccggagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 7 - DNA, 2531 bp, Artificial Sequence
  CBA GBA1 WPRE expression construct
ccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggtaccggatcctctagagtcgactccggagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 8 - DNA, 2914 bp, Artificial Sequence
  CAG GBA1 WPRE expression construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggtaccggatcctctagagtcgactccggagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 9 - DNA, 5961 bp, Artificial Sequence
  pAAV.CBA.hGBA1.WPRE.hGHpA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggtaccggatcctctagagtcgactccggagccacc

SEQ ID NO: 10 - DNA, 6344 bp, Artificial Sequence
  pAAV.CAG.hGBA1.WPRE.hGHpA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggtaccggatcctctagagtcgactccggagccacc

SEQ ID NO: 11 - DNA, 6186 bp, Artificial Sequence
  pAAV.hSYN.hGBA1.WPRE.hGHpA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaagggtaccggatcctctagagtcgactccggagccacc

SEQ ID NO: 12 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 13 - DNA, 1611 bp, Artificial Sequence
  Codon optimised sequence encoding GBA1
atggagttcagctccccttctagggaggagtgccctaagccactgtctcgcgtgagcatcatggcaggaagcctgaccggactgctgctgctgcaggccgtgtcctgggcatctggagcaaggccctgcatccctaagtccttcggctactctagcgtggtgtgcgtgtgcaacgccacctattgtgactccttcgatccccctacctttcccgccctgggcacattttctcggtacgagagcacaagatccggccggagaatggagctgtctatgggccccatccaggccaatcacaccggaacaggactgctgctgaccctgcagcctgagcagaagttccagaaggtgaagggctttggaggagcaatgacagacgcagccgccctgaacatcctggccctgagcccacccgcccagaatctgctgctgaagtcttacttcagcgaggagggcatcggctataacatcatccgggtgccaatggcctcctgtgacttttctatcagaacctacacatatgccgatacccccgacgatttccagctgcacaatttttccctgcctgaggaggatacaaagctgaagatcccactgatccacagggccctgcagctggcacagaggcctgtgagcctgctggcatccccatggacctctcccacatggctgaagaccaacggcgccgtgaatggcaagggaagcctgaagggacagccaggcgacatctaccaccagacatgggcccggtatttcgtgaagtttctggatgcctacgccgagcacaagctgcagttctgggcagtgaccgcagagaacgagccatctgccggactgctgagcggatatccattccagtgcctgggctttacacccgagcaccagagggactttatcgcaagggatctgggacctaccctggccaactccacacaccacaatgtgcggctgctgatgctggacgatcagagactgctgctgccccactgggccaaggtggtgctgaccgaccctgaggccgccaagtacgtgcacggcatcgccgtgcactggtatctggatttcctggcaccagcaaaggccaccctgggagagacacacaggctgttccccaacaccatgctgtttgccagcgaggcctgcgtgggctccaagttttgggagcagtccgtgcggctgggatcttgggacagaggcatgcagtacagccactccatcatcaccaatctgctgtatcacgtggtgggctggacagactggaacctggccctgaatcctgagggcggcccaaactgggtgagaaatttcgtggatagccccatcatcgtggacatcaccaaggatacattctacaagcagcctatgttttatcacctgggccacttctctaagtttatcccagagggaagccagagggtgggactggtggcaagccagaagaacgacctggatgccgtggccctgatgcacccagatggctccgccgtggtggtggtgctgaatcgctcctctaaggacgtgcccctgaccatcaaggatcctgccgtgggctttctggagaccatcagccccggctactccatccacacatatctgtggaggcgccagtga

SEQ ID NO: 14 - DNA, 1611 bp, Artificial Sequence
  Codon optimised sequence
atggagttctcttctccatctcgcgaggagtgccctaagccactgagtcgagtgtctatcatggccggcagtctgacaggactcctcctcctacaagctgtgtcttgggccagtggcgcccggccatgcatcccaaagtctttcgggtactctagcgtggtgtgcgtgtgcaacgctacatactgcgactctttcgacccacctacattccccgccctgggaaccttcagccgatacgagtctacacggtccgggcggcgcatggagctgtcaatggggccaattcaggctaaccacaccggaacaggactcctgctgacattacaacccgagcagaagttccagaaggtgaaggggttcggcggagctatgaccgacgctgccgctctgaacattctggccctgtccccacccgctcagaacctgctcctcaagtcttacttcagcgaggagggaatcgggtacaacattattagagtgcctatggcttcttgcgacttcagcatccgcacatacacatacgccgacacccctgacgacttccagctgcacaacttcagcctcccagaggaggacacaaagctcaagatcccactgattcaccgcgctttacagctcgctcagaggccagtgtctctgctcgcttccccttggacatctccaacatggttaaaaacaaacggcgccgtgaacggaaaggggtccctgaaggggcagcccggagacatttaccaccagacatgggccaggtacttcgtgaagttcctcgacgcttacgctgagcacaagctccagttctgggccgtgaccgccgagaacgagcctagcgccggcctgctgtccggataccctttccagtgcctcggattcacaccagagcaccagcgggacttcattgctagagacctcgggccaacactcgctaactctacccaccacaacgtgagactgctgatgctcgacgaccagcgcctcctgctgcctcactgggctaaggtggtgctcacagaccccgaggccgccaagtacgtgcacggaattgccgtgcactggtatctcgacttcctcgccccagctaaggctacactcggcgagacccacaggctgttccctaacacaatgctgttcgccagcgaggcttgcgtgggaagcaagttctgggagcagtccgtgagactcggatcttgggaccgcggaatgcagtactctcactctatcattacaaacctcctgtaccacgtggtgggatggaccgactggaacctggctctcaaccccgagggcggcccaaactgggtgagaaacttcgtggacagccctattattgtggacattacaaaggacaccttctacaagcagcctatgttctaccacctcgggcacttcagcaagttcatccccgagggatctcagagagtgggactggtggcctctcagaagaacgacctcgacgctgtggctctgatgcaccctgacgggtcggccgtggtggtggtgctcaaccgatctagcaaggacgtgccactgacaattaaggaccccgccgtggggttcctggagacaattagccccggatactccattcacacatacctgtggcgcaggcagtga

SEQ ID NO: 15 - DNA, 1611 bp, Artificial Sequence
  Codon optimised sequence encoding GBA1
atggagtttagcagccctagcagagaagagtgccctaagcctctgtctcgtgtgagcatcatggccggctccttaaccggattattactgctgcaagctgtgtcttgggcttccggcgctaggccttgtattcccaagtccttcggctactcctccgtggtgtgcgtctgtaatgccacctactgtgacagcttcgatccccccacattccccgctctgggcacattttctcgttacgaaagcactcgtagcggaaggaggatggagctgagcatgggccctattcaagctaaccacaccggcaccggtttattactgactttacaacccgagcagaagttccagaaggtgaagggctttggaggcgccatgacagacgccgccgctttaaatattctggctttaagccctcccgcccaaaatctgctgctgaagtcctacttctccgaggaaggcatcggctacaacatcattagggtccctatggcctcttgtgacttcagcattaggacctacacctacgccgacacccccgacgacttccagctgcacaacttctctttacccgaggaggatacaaagctgaagatccctttaatccatcgtgccctccagctggctcagaggcccgttagcttattagcctccccttggacatcccctacttggctgaaaaccaacggcgctgtcaacggaaagggctctttaaagggacagcccggcgacatttaccaccagacatgggctaggtacttcgtgaagtttttagacgcttatgccgaacacaagctgcagttctgggccgtgaccgctgagaacgagccttccgccggtttactgtccggataccccttccaatgtctgggattcacccccgaacaccagagggacttcatcgccagagatctgggccccacactggccaattccacacaccacaacgtgagactgctcatgctggacgaccagaggttattactccctcactgggccaaagtggtgctgaccgaccccgaagctgccaagtatgtgcatggaatcgccgtgcactggtatctggactttctggcccccgccaaggctacactgggcgagacccacagactgttccctaacaccatgctcttcgccagcgaagcttgcgtgggaagcaagttctgggagcaatccgtgaggctgggcagctgggatcgtggcatgcagtactcccactccattatcacaaatctgctctaccacgtcgtgggatggaccgattggaatttagctctgaaccccgagggaggccccaattgggtgaggaacttcgtggattcccccatcatcgtcgatatcaccaaggacacattctacaagcagcccatgttctaccatttaggccactttagcaagttcatccccgagggaagccagagagtgggtttagtggcctcccagaagaacgatctggacgccgtcgccctcatgcatcccgatggcagcgctgtggtcgtcgtgctgaatagaagctccaaagacgtccctttaaccatcaaggaccccgccgtgggctttctggaaaccatctcccccggttatagcatccacacctatttatggagaagacagtaa

SEQ ID NO: 16 - DNA, 1611 bp, Artificial Sequence
  Codon optimised sequence encoding GBA1
atggaattttctagcccctccagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcccgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtttctctgctggctagcccctggacatctcccacctggctgaaaacaaatggcgccgtgaatggcaagggcagcctgaaaggccaacctggcgatatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgccagagatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggccagccagaagaatgatctggacgccgtggctctgatgcaccctgatggatctgctgtggtggtggtcctgaaccggtccagcaaagatgtgcccctgaccatcaaggatcccgccgtgggattcctggaaacaatcagccctggctactccatccacacctacctgtggcggagacagtag

SEQ ID NO: 17 - DNA, 5961 bp, Artificial Sequence
  pAAV.CBA.hGBA1.WPRE.hGHpA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggtaccggatcctctagagtcgactccggagccacc

SEQ ID NO: 18 - DNA, 6359 bp, Artificial Sequence
  pAAV.CAG.hGBA1.WPRE.hGHpA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtaccggatcctctagagtcgactccggagccacc

SEQ ID NO: 19 - DNA, 6185 bp, Artificial Sequence
  pAAV.hSYN.hGBA1.WPRE.hGHpA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatcctctagagtcgactccggagccacc

SEQ ID NO: 20 - DNA, 2290 bp, Artificial Sequence
  CAG GBA1 expression construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtaccggatcctctagagtcgactccggagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 21 - DNA, 2913 bp, Artificial Sequence
  CAG GBA1 WPRE expression construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtaccggatcctctagagtcgactccggagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg